(19) United States
(12) Patent Application Publication
(10) Pub. No.: US 2021/0077741 A1
(43) Pub. Date: Mar. 18, 2021

(54) Injection Device with a Preselector

(71) Applicant: Sanofi-Aventis Deutschland GMBH, Frankfurt am Main (DE)

(72) Inventors: Michael Helmer, Frankfurt am Main (DE); Steffen Raab, Frankfurt am Main (DE); Maurice Toporek, Frankfurt am Main (DE)

(21) Appl. No.: 16/761,341

(22) PCT Filed: Nov. 5, 2018
(86) PCT No.: PCT/EP2018/080081

§ 371 Date: May 4, 2020

(30) Foreign Application Priority Data
Nov. 7, 2017 (EP) 17200317.0

(51) Int. Cl. A61M 5/315; A61M 5/28; A61M 5/31

(52) U.S. Cl. CPC A61M 5/31566; A61M 5/3155; A61M 5/28; A61M 5/3129; A61M 2005/3154; A61M 2205/3553; A61M 2205/585; A61M 2005/3126; A61M 2205/502

(57) ABSTRACT

An injection device for setting and injecting a dose of a medicament comprises a housing) extending along an axial direction (z), a dose setting mechanism arranged in the housing, a dose dial displaceable relative to the housing for setting of the dose, a dose tracker (50; 150) operably connectable to the dose dial (12), the dose tracker is at least one of translationally or rotationally displaceable relative to the housing during setting of a dose, wherein a positional state of the dose tracker relative to the housing is indicative of a size of the dose, and a preselector configured to define a maximum dose positional state of the dose tracker relative to the housing, and a first marker provided on one of the dose tracker and the preselector and configured to indicate the positional state of the dose tracker relative to the preselector.

### CROSS REFERENCE TO RELATED APPLICATIONS

The present application is the national stage entry of International Patent Application No. PCT/EP2018/080081, filed on Nov. 5, 2018, and claims priority to Application No. EP 17200317.0, filed on Nov. 7, 2017, the disclosures of which are incorporated herein by reference.

### DESCRIPTION

The present disclosure relates in one aspect to injection device, such as a pen-type injector for setting and dispensing of a dose of a medicament. In particular, the disclosure relates to an injection device comprising a preselector configured to preselect or limit a maximum dose that can be a set and dispensed by the injection device. In another aspect the disclosure relates to an add-on device for use with an injection device in order to monitor and/or to control operation of the injection device

### BACKGROUND

Injection devices for setting and dispensing a single or multiple doses of a liquid medicament are as such well-known in the art. Generally, such devices have substantially a similar purpose as that of an ordinary syringe.

Injection devices, in particular pen-type injectors have to meet a number of user-specific requirements. For instance, with patient's suffering chronic diseases, such as diabetes, the patient may be physically infirm and may also have impaired vision. Suitable injection devices especially intended for home medication therefore need to be robust in construction and should be easy to use. Furthermore, manipulation and general handling of the device and its components should be intelligible and easy understandable. Moreover, the dose setting as well as dose dispensing procedure must be easy to operate and has to be unambiguous.

Typically, such devices comprise a housing including a particular cartridge holder, adapted to receive a cartridge at least partially filled with the medicament to be dispensed. Such devices further comprise a drive mechanism, usually having a displaceable piston rod which is adapted to operably engage with a piston of the cartridge. By means of the drive mechanism and its piston rod, the piston of the cartridge is displaceable in a distal direction or dispensing direction and may therefore expel a predefined amount of the medicament via a piercing assembly, which is to be releasably coupled with a distal end section of the housing of the injection device.

The medicament to be dispensed by the injection device is provided and contained in a multi-dose cartridge. Such cartridges typically comprise a vitreous barrel sealed in a distal direction by means of a pierceable seal and being further sealed in proximal direction by the piston. With reusable injection devices an empty cartridge is replaceable by a new one. In contrast, injection devices of disposable type are to be discarded when the medicament in the cartridge has been dispensed or used-up.

For some applications it can be advantageous to limit a maximum size of a dose that can be dispensed or expelled from the cartridge. Then, unintended overdosing of the medicament could be prevented.

### Objects

It is therefore an object of the present disclosure to provide an injection device with an increased patient safety and which comprises a mechanism that prevents unintended overdosing of a medicament. The injection device should provide a limited capability to set and to dispense doses of different sizes. The injection device should at least temporally provide setting and dispensing of only one or a few differently sized doses. In particular, the injection device should be configured to allow and enable setting and dispensing of only a few, e.g. of two, three or four differently sized doses of the medicament. Moreover, indication or displaying of a size of a dose of appropriate size should be simplified. Indicating or displaying of a size of a dose should be unequivocal, intuitive and straightforward even for patients suffering side effects such as impaired vision.

It is a further aim to provide an add-on device configured for attachment to such an injection device or being configured to be coupled to such an injection device in a data transferring or information transferring way

### SUMMARY

In one aspect there is provided an injection device for setting and injecting a dose of a medicament. The injection device comprises a housing as well as a dose setting mechanism which is arranged in the housing. The injection device further comprises a dose dial displaceable relative to the housing for setting of a dose. The dose dial may be rotatably supported in or on the housing. It may be rotationally supported at a proximal end section of the housing. The dose dial is user actuatable. Hence, a user may grip and rotate or displace the dose dial relative to the housing for setting or selecting of a dose of variable size. The degree of displacement of the dose dial relative to the housing is indicative to the size of the dose.

Typically, the housing is also sized and configured to accommodate a container filled with the liquid medicament. The container may comprise a cartridge with a tubular shaped barrel and sealed in proximal direction by means of a bung displaceably arranged inside the barrel for expelling the dose of the medicament via a distal end of the barrel. The distal end of the barrel may be sealed by a pierceable seal, such as a septum.

The injection device and/or its dose setting mechanism further comprises a dose tracker. The dose tracker is operably connectable to the dose dial at least during setting of a dose. The dose tracker is at least one of translationally or rotationally displaceable relative to the housing during setting of a dose. A positional state of the dose tracker, i.e. a position and/or an orientation of the dose tracker relative to the housing is indicative of a size of the dose actually set. Hence, in the present context a ‘positional state’ of a component includes a position of the component and an angular orientation of the component relative to another component or relative to the housing. A positional state of the dose tracker reflects for instance at least one or both of the geometric position as well as an angular orientation of the dose tracker relative to the housing of the injection device.

The injection device further comprises a preselector configured to define a maximum dose positional state of the dose tracker relative to the housing. The preselector is configured to vary a maximum dose positional state of the dose tracker. The maximum dose positional state of the dose tracker defines and coincides with a maximum size of a dose that can be set and dispensed with the injection device. By means of the preselector the maximum dose positional state can be changed between at least two states that reflect different maximum dose sizes of the injection device. The preselector may be configured to block a displacement of the dose tracker beyond the maximum dose positional state. Depending on the configuration of the preselector the maximum displacement of the dose dial and hence of the dose tracker for setting of a dose can be varied.

The injection device further comprises at least a first marker provided on one of the dose tracker and the preselector. The first marker is configured to indicate the positional state of the dose tracker relative to the preselector. During dose setting the preselector is typically immobilized relative to the housing. Since the positional state of the dose tracker relative to the housing is indicative of a size of a dose the same is then also valid for the preselector. Before setting of a dose or before conducting a dose setting procedure the preselector is configured to define a maximum dose positional state.

The position or configuration of the preselector is known. Since the preselector is immobile or fixed to the housing at least during setting of a dose by detecting a position or orientation of the first marker on one of the dose tracker and the preselector a positional state of the dose tracker relative to the preselector can be determined. This relative position or orientation of the first marker and hence of the dose tracker relative to the preselector indicates whether the dose tracker has reached the maximum dose positional state or whether the dose tracker has to be displaced further relative to the housing in order to reach the maximum dose positional state.

With at least a first marker a relative position and/or orientation of the dose tracker relative to the preselector can be determined. In this way a conventional display to indicate or to visualize a momentary positional state of the dose tracker and/or of the dose dial can be substituted by the first marker and its ability to distinguish only between two positional states of the dose tracker in relation to the preselector.

The preselector is configured to define one of a plurality of generally possible maximum dose positional states of the dose tracker. The configuration or manipulation of the preselector defines one of a variety of maximum dose positional states. After a definition of one maximum dose positional state on the basis of the preselector the dose dial is user actuatable in order to displace the dose tracker relative to the housing until it reaches the maximum dose positional state.

Dispensing of a dose is only to be conducted when the dose tracker is in the maximum dose positional state. It is therefore sufficient and easily understandable for a user when an indication is given on or with regard to the injection device that either a maximum dose positional state has been reached or not. In case the maximum dose positional state has not yet been reached the user is encouraged to dial the dose dial further until the dose tracker reaches the maximum dose positional state. Upon reaching the maximum dose positional state the injection device is configured to indicate the maximum dose positional state on the basis and by means of the first marker.

The first marker provided on one of the dose tracker and the preselector enables and provides a rather simple, intuitive and easily understandable approach to provide an indicator assembly to visualize a correct setting of a dose of predefined size. A conventional display, e.g. in form of a dosage window in a sidewall of a housing of an injection device, through which window a section of a rotatable number sleeve is visible can be substituted. The complexity of the injection device and of its operation can be therefore reduced.

According to another example the preselector is at least one of translationally or rotationally displaceable relative to the housing between at least two preselection positional states. The preselector is lockable relative to the housing in one of the two preselection positional states. A first preselection positional state may represent and define a first maximum dose positional state. A second preselection positional state of the preselector defines a second maximum dose positional state. The first and the second maximum dose positional states are different.

The preselector is typically only one of translationally or rotationally displaceable relative to the housing. It may comprise a slider being slidably displaceable along a longitudinal axis of the elongated housing. The elongated housing may comprise a tubular or cylindrical shape. It may extend along an axial direction. In the following, the terms axial and longitudinal are used as synonyms. It is also conceivable that the preselector is rotationally displaceable relative to the housing with the longitudinal axis as an axis of rotation. Also, the preselector can be displaced along a sidewall of the housing in a circumferential or tangential direction. Hence, the preselector may comprise a slider being either slidably displaceable relative to the housing along the axial direction and/or along a tangential or circumferential direction.

The preselector is displaceable along a displacement path. The at least two preselection positional states of the preselector are separated along the displacement path. In any of the at least two preselection positional states the preselector is lockable or fixable to the housing. In this way the preselector is stationary and hence fixed to the housing during setting of a dose. After completion of a dose dispensing procedure and if desired the preselector may be displaced to another of the at least two preselection positional states.

With the preselector, the operability and function of the dose dial can be reduced. With the preselector a maximum size of a dose that can be dialed or selected by means of the dose dial can be predefined and hence limited to a given maximum. During setting or selecting of a dose the dose dial can be displaced relative to the housing to displace the preselector to the maximum dose positional state. Since the maximum dose positional state is defined by the preselector it cannot be overruled by the dose dial or dose tracker.

In this way the dose setting procedure is divided into two separate steps. In a first step and by making use of the preselector a maximum dose positional state is defined for the injection device. In a second step thereafter the dose dial is user actuatable to move the dose tracker until it reaches the maximum dose positional state. The separation of the dose setting procedure into these two steps by means of two separate components, namely by the preselector and the dose dial or dose tracker has the benefit that the operability of the injection device in terms of dose setting can be restricted to a limited number of dose sizes.

A caregiver or medical staff may use or configure the preselector to define a maximum dose positional state for the dose tracker. Control of the preselector may be prohibited for the end user or patient. The preselector may be locked in one of the at least two preselection positional states in such a way that the end user or patient is unable to unlock and to modify the preselection positional state of the preselector. In this way the end user or patient is only given the possibility to set a dose by making use of the dose dial, which according to the interaction between the preselector and the dose tracker is only enabled and configured to displace the dose tracker at maximum to the maximum dose positional state. Overdosing can be thus effectively prevented.

Use of the injection device by a patient becomes safer since the injection device is preconfigured for only one predefined dose size. By means of the preselector the injection device originally configured as a variable dose size injection device can be transferred or transformed into a fixed dose injection device preconfigured to set and to dispense numerous doses of a medicament of a predefined size.

On demand and without the necessity to change the injection device a dose size can be modified by reconfiguring the preselector and/or by translationally or rotationally displacing the preselector to a different preselection positional state.

According to another example the injection device comprises an indicator assembly that is configured to indicate when the dose tracker reaches the maximum dose positional state. The indicator assembly is typically configured to visually indicate the coincidence of a momentary state of the dose tracker with the maximum dose positional state. However, the indicator assembly is not limited to visual indicator assemblies. Generally, the indicator assembly may comprise an audible or tactile indicator that produces a sound or that starts to vibrate in response to the dose tracker reaching the maximum dose positional state.

The indicator assembly is typically configured to indicate the relative positional state between the preselector and the dose tracker. If the at least first marker is provided on the dose tracker the indicator assembly or at least a portion thereof is provided on the preselector. In another example, wherein the at least first marker is provided on the preselector the indicator assembly or at least a portion thereof is provided on the dose tracker. The indicator assembly is configured to cooperate or to interact with the at least first marker. One of the dose tracker and the preselector is typically provided with the at least first marker wherein the other one of the dose tracker and the preselector is provided with the indicator or at least a portion thereof. In this way the indicator and that at least first marker are configured to provide an indication representing a relative position of the preselector relative to the dose tracker or vice versa.

In another example the first marker is located on the dose tracker. The indicator assembly is configured to reveal the first marker on the dose tracker when the dose tracker reaches the maximum dose positional state. The dose tracker may comprise a longitudinally extending component of the dose setting mechanism. The dose tracker may comprise a dose sleeve rotatable inside the housing, axially displaceable inside the housing or being threadedly engaged inside the housing. The preselector may comprise a button, a dial or a slider displaceable between the at least two preselection positional states.

The dose tracker and the preselector may be arranged in a radially overlapping way on or inside the housing of the injection device. During setting of a dose the dose tracker is typically displaceable relative to the preselector and relative to the housing of the injection device. Typically, the indicator assembly is located on or is attached to one of the preselector and the housing of the injection device. The housing, the preselector and/or the indicator assembly may cover the first marker that may be provided on an outside surface of the dose tracker as long as the dose tracker has not yet reached the maximum dose positional state. The indicator assembly, the preselector and the dose tracker may be configured to reveal the at least first marker provided on the dose tracker only when the dose tracker reaches the maximum dose positional state. The indicator assembly may be provided by the preselector and/or by the housing of the injection device.

In a further example the indicator assembly is integrated into the preselector. The indicator assembly comprises an aperture extending through the preselector. Here, the preselector may cover at least a portion of the dose tracker. The preselector may also cover at least a portion of a sidewall of the housing of the injection device. The first marker that is provided on an outside surface of the dose tracker is discernible and visible through the aperture of the indicator assembly when the dose tracker reaches the maximum dose positional state.

It is conceivable that the preselector is located in a recess on the sidewall of the housing of the injection device such that a plurality or a major portion of the recess in the sidewall, which recess may be a through opening in the sidewall, is covered by the preselector. Typically, the preselector is displaceable relative to the housing along the displacement path. The dose tracker may be also displaceable at least parallel or in the direction of the elongation of the displacement path of the preselector. In the maximum dose positional state the position of the dose tracker relative to the preselector is always the same irrespective on the position of the preselector relative to the housing. In this way an aperture extending through the preselector and revealing at least a portion on an outside surface of the dose tracker is generally sufficient to indicate whether the dose tracker has reached a maximum dose positional state or not.

The integration of the indicator assembly into the preselector is of particular benefit since there is no need for any further mechanical or electronic components to indicate whether a maximum dose positional state of the dose tracker has been reached or not. The preselector may therefore provide a twofold or double function. On the one hand it is configured to define a maximum dose positional state of the dose tracker. On the other hand the preselector is enabled to visualize whether the dose tracker has reached the predefined maximum dose positional state or not.

In another example the indicator assembly comprises a magnifying lens arranged in the aperture. A magnifying or magnification lens enables an enlarged view of the at least first marker provided on the dose tracker when the at least first marker shows up in the aperture of the preselector. A magnifying lens provides a good, intuitive and easy legibility of the at least first marker.

The at least first marker may comprise any visual symbol, such as a character, a number or a colored area. For instance, the at least first marker may comprise a green colored surface section on the outside surface of the dose tracker. Other portions on the outside surface of the dose tracker, such as a dedicated second surface portion may comprise a different color, such as a red color. As long as a red color shows up in the aperture of the preselector this is an indication to a user, that the maximum dose positional state of the preselector has not yet been reached. The user is hence encouraged to dial the dose dial further in a dose incrementing direction until the green colored first marker shows up in the aperture of the preselector. This would be a clear and unequivocal indication to the end user or patient that the maximum dose positional state has been reached and that the injection procedure, hence dispensing of a dose actually set may commence.

In another embodiment the dose tracker comprises at least one tracking stop feature wherein the preselector comprises at least one preselector stop feature. The preselector stop feature is configured to engage with the at least one tracking stop feature to block and to prevent a displacement of the dose tracker beyond the maximum dose positional state. Insofar the dose tracker and the preselector are mechanically engageable.

The positional state of the preselector, hence the preselection positional state of the preselector relative to the housing provides an end stop and a barrier for the dose tracker travelling in a dose incrementing direction. As soon as the dose tracker mechanically engages with the preselector any further displacement of the dose tracker relative to the housing in a dose increasing direction is effectively blocked. Here, the preselector behaves and acts as a limiter that is configured to limit a dose setting displacement of the dose tracker and hence of the dose dial in a dose incrementing direction relative to the housing.

As soon as the dose tracker reaches the maximum dose positional state its tracking stop feature engages and hence abuts with the preselector stop feature that is at least temporally locked to a predefined longitudinal or axial portion of the sidewall of the housing in accordance to the respective preselection positional state of the preselector. The preselector stop feature and the tracking stop feature may comprise mutually corresponding stop faces, e.g. extending in circumferential and/or radial direction so as to engage axially. Alternatively or additionally the preselector stop feature and the tracking stop feature may comprise mutually corresponding stop faces extending in axial direction and radial direction so as to engage circumferentially.

When configured to engage axially, the mutual engagement of the preselector stop feature and the tracking stop feature provides an axial stop thereby impeding and blocking a longitudinal or axial translation of the dose tracker beyond the maximum axial dose positional state.

When configured to engage circumferentially or tangentially, the mutual engagement of the preselector stop feature and the tracking stop feature provides a rotational stop, thereby impeding and blocking a rotation of the dose tracker relative to the preselector and hence relative to the housing beyond a predefined maximum rotational dose positional state.

The selector stop feature and the limiter stop feature may comprise both, longitudinally and circumferentially extending stop faces configured to mutually engage. In this way a longitudinal as well as a rotational displacement of the dose tracker relative to the housing and hence relative to the preselector can be effectively prevented in a twofold manner. This provides an even improved stop configuration and mechanical abutment for the dose tracker.

In another example the first marker is provided on the dose tracker wherein a second marker is provided on the preselector. In this example the first marker of the dose tracker is subject to a displacement relative to the housing during a dose setting procedure. The second marker which is provided on the preselector is subject to a displacement relative to the housing during a preselection procedure. During dose setting only the dose tracker is moveable relative to the housing while the preselector is stationary relative to the housing. The dose tracker is displaceable relative to the housing until the first marker overlaps, aligns or coincides with the second marker of the preselector. Mutually overlapping, mutually aligning or mutually coinciding first and second markers may be a direct indication to the user or patient that the maximum dose positional state of the preselector has been reached.

The first and the second markers may comprise mutually corresponding symbols or signs, such as mutually corresponding arrows or the like pointers providing a clear and unequivocal appearance when aligning or overlapping mutually thus indicating that the dose tracker has reached the maximum dose positional state.

In another example the indicator assembly is configured to determine the positional state of the dose tracker on the basis of the first marker and wherein the indicator assembly is configured to determine the preselection positional state of the preselector on the basis of the second marker. For this it is not required that the first and the second markers mutually align, overlap or coincide. Rather, the indicator assembly is configured to separately determine the positional state of the first marker and to determine the positional state of the second marker. The acquired positional states of the first and the second markers may be further evaluated or processed by the indicator assembly in order to determine whether the maximum dose positional state has been reached or not.

For this the indicator assembly may comprise at least a first position detector and a second position detector, wherein the first position detector is configured to determine or to measure a positional state of the first marker and wherein the second position detector is configured to determine or to measure a positional state of the second marker. In this way the indicator assembly is configured to separately and independently determine a positional state of the first marker and of the second marker and hence of the dose tracker and the preselector, respectively.

This provides a multitude of geometric variations for the specific implementation of the dose tracker and the preselector. The preselector and the dose tracker do not have to be arranged in an overlapping or partially overlapping configuration. The preselector may be located at a totally different position on or in the housing of the injection device compared to the position of the dose tracker. This allows and enables a universal and variable design of the injection device.

According to another example the indicator assembly is configured to compare the positional state of the dose tracker with the preselection positional state of the preselector. This comparison is typically based on previously acquired or detected positions of the first marker, e.g. provided on the dose tracker and on the position or positional state of the second marker provided on the preselector.

In particular, the indicator assembly may comprise an electronic indicator assembly having at least a processor and a display, preferably an electronic display. The processor may be configured to compare the positional state of the dose tracker as detected by way of of the first marker and on the basis of the first position detector with the preselection positional state of the preselector as detected by the second position detector by way of of the second marker that is provided on the preselector.

First and second position detectors may be provided in or on the housing of the injection device. The first position detector may be configured to determine a positional state of the first marker relative to the housing. Likewise, the second position detector may be configured to determine the positional state of the second marker relative to the housing. The positional states of the first marker and the second marker as detected or measured by first and second position detectors can be mutually compared in order to extract and in order to determine a positional state of the dose tracker relative to the preselector which is indicative of whether the maximum dose positional state of the preselector has been reached or not.

In another example the indicator assembly comprises a processor and an electronic display. The processor and the electronic display are configured to visualize at least one of the positional state of the dose tracker and the preselection positional state of the preselector. Additionally or alternatively, the processor and the electronic display are configured to visualize the positional state of the dose tracker relative to the preselection positional state of the preselector. Since the positional state of the preselector governs and defines the maximum dose positional state of the dose tracker it is generally sufficient to visualize the positional state of the dose tracker relative to the preselection positional state of the preselector.

As long as the dose tracker is not in the maximum dose positional state, the indicator assembly, the processor and the electronic display are configured to provide an indication that the maximum dose positional state has not been reached. As soon and in response to a detection that the dose tracker has reached a positional state relative to the preselector that corresponds to its maximum dose positional state the processor and the electronic display are configured to indicate that the preselector has reached the predefined maximum dose positional state.

Apart from that the processor and the electronic display may also provide information regarding the preselection positional state and the momentary positional state of the dose tracker. In this way, additional information is provided to the user during a dose setting procedure.

According to a further example the indicator assembly comprises a communication unit connected to the processor to exchange electronic data with a remote electronic device. A communication unit is typically implemented as a wireless communication unit. It may be based on conventional radio-frequency wireless transmission standards, such as one of the communication protocols: near field communication NFC, RFID or IEEE 802.11.

The communication unit enables data transmission and data exchange with a remote electronic device. The remote electronic device may comprise or may be provided by a smartphone or some other portable electronic device. In this way, operation of the indicator assembly and data acquired by the indicator assembly can be transmitted to the remote electronic device. In addition, the communication unit may enable and control a reconfiguration of the preselector.

The presently described concept of the injection device is not only limited to purely mechanically operated injection devices, such as mechanically implemented injection pens. The preselector and the injection device may be electromechanically implemented and may be remote controlled by means of the communication module of the indicator assembly.

Here, the remote electronic device may reconfigure the preselector via the communication unit of the indicator assembly. In this way and by means of the communication unit the electronically or electromechanically implemented preselector can be switched to one of the at least two preselection positional states. This provides a further approach to hinder the patient or end user to manipulate the preselector. The reconfiguration of the preselector via the communication unit of the injection device may be password protected or may be encoded. A reconfiguration or manipulation of the electromechanically implemented preselector requires knowledge of a respective electronic authorization or code. Typically, the remote electronic device comprises a software to enable communication with the communication unit in order to reconfigure the preselector and/or the indicator assembly.

The communication unit, the processor and the indicator assembly may be provided as integral components of the injection device that are undetachably connected to the housing of the injection device. In other embodiments a detachable solution is provided, wherein the indicator assembly, the communication unit and/or the processor is provided as a part of an add-on device detachably connectable to the housing of the injection device.

According to a further example the injection device comprises a piston rod and a cartridge. The cartridge comprises a barrel which is filled with a medicament and which is sealed by a bung or a piston. The bung or piston seals a proximal end of the barrel. The bung or the piston is axially displaceable relative to the barrel by means of the piston rod. The piston rod is displaceable in distal direction, hence towards a distal dispensing end of the injection device. An advancing and expelling motion of the piston rod can be induced and controlled by a drive mechanism. The drive mechanism may be configured to drive the piston rod in distal direction in accordance to the positional state of the dose tracker as previously set during a dose setting procedure. During dose dispensing and while the piston rod advances in distal direction the dose tracker returns from a maximum dose positional state to a zero dose positional state.

The injection device may comprise a drive mechanism which is purely mechanically implemented, wherein a driving force for displacing the piston rod in distal direction is exclusively provided by the operator, e.g. by a thumb or a finger of the operator or user of the device. The injection device is not limited to all mechanically implemented injection devices. The injection device may comprise an electric drive or may comprise at least a mechanical energy reservoir providing at least a portion of a dispensing force required for displacing the piston rod in distal direction during expelling of a dose from the cartridge.

According to another aspect an add-on device that is configured for attachment to an injection device is provided. Alternatively, the add-on device may be configured to be coupled to the injection device. The add-on device may be coupled to the injection device for exchanging electronic data. The add-on device is particularly configured for attachment to or for data transferring coupling with an injection device as described above. The add-on device comprises at least a remote indicator assembly that is configured to indicate a positional state of the dose tracker of the injection device relative to the preselector of the injection device. In this way, the injection device may be void of an own indicator assembly. The remote indicator assembly of the add-on device may provide and substitute the indicator assembly as described above in connection with the injection device. This applies when the add-on device is correctly attached to the housing of the injection device or when the add-on device is correctly coupled to the injection device, e.g. in a data transferring way.

The add-on device is particularly configured for use with disposable injection devices that are intended to be discarded after the content of the cartridge has been used or expelled. Typically the add-on device may comprise a fastener to engage with a counter fastener of the housing of the injection device. In this way the add-on device can be positioned at a well-defined location on or at the housing of the injection device. The add-on device may precisely determine a positional state of the dose tracker relative to at least one of the preselector and the housing of the injection device. Additionally or alternatively the add-on device and the remote indicator assembly may determine a positional state of the preselector relative to the dose tracker and/or relative to the housing of the injection device. Swapping and exchanging one injection device by another injection device only requires to detach the add-on device from an initial injection device and to attach the add-on device to the new injection device.

In a further example the remote indicator assembly of the add-on device comprises at least one of a first position sensor and a second position sensor. Here, the first position sensor is particularly configured to determine the positional state of the dose tracker. The second position sensor is particularly configured to determine the preselection positional state of the preselector. Insofar the remote indicator assembly may be configured to separately determine and to separately measure a positional state of the dose tracker and/or a preselection positional state of the preselector independently from each other.

As described above with regard to the electronically implemented indicator assembly also the remote indicator assembly of the add-on device may be configured to compare the positional state of the dose tracker with the preselection positional state of the preselector. The first position sensor may be particularly adapted to identify or to determine the position of the first marker that is provided on the dose tracker. The second position sensor may be likewise configured to determine or to detect a positional state of the second marker that is provided on the preselector.

In general, the remote indicator assembly may be identically shaped and configured compared to the electronically implemented indicator assembly of the injection device. Insofar any features, benefits and advantages described above with regard to the indicator assembly of the injection device are also valid and disclosed for the remote indicator assembly of the add-on device.

Accordingly and in another example of the add-on device the remote indicator assembly comprises a processor and an electronic display. The processor and the electronic display are configured to visualize at least one of the positional state of the dose tracker and the preselection positional state of the preselector. Additionally or alternatively the processor and the electronic display of the remote indicator assembly are configured to visualize the positional state of the dose tracker relative to the preselection positional state of the preselector.

The processor and the electronic display may be configured to visualize the absolute positional states of at least one or both of the dose tracker and the preselector simultaneously or concurrently. It is also conceivable, that the indicator assembly or the remote indicator assembly of the injection device and/or of the add-on device provide an alternating visualization of the actual or momentary positional state of the dose tracker and the preselection positional state of the preselector. The processor and the electronic display may be configured to provide alternative illustrations of the momentary preselection positional state and the actual positional state of the dose tracker. Both indications, e.g. provided in form of numbers or confirmation symbols may be provided next to each other on the electronic display or in an overlapping but temporally alternating way.

The first and second position sensors for determining a positional state of the dose tracker and/or for determining the preselection positional state of the preselector may comprise at least one of electromechanical contact switches, a light detector, such as a photodiode in connection with a light source and/or a transmissive or reflective pattern on the dose tracker and the preselector, respectively, a magnetic sensor in combination with a magnetic encoding on at least one of the dose tracker and the preselector or an electrically conductive structure on at least one of the dose tracker and the preselector.

In the present context the term ‘distal’ or ‘distal end’ relates to an end of the injection device that faces towards an injection site of a person or of an animal. The term ‘proximal’ or ‘proximal end’ relates to an opposite end of the injection device, which is furthest away from an injection site of a person or of an animal.

The term “drug” or “medicament”, as used herein, means a pharmaceutical formulation containing at least one pharmaceutically active compound,

wherein in one embodiment the pharmaceutically active compound has a molecular weight up to 1500 Da and/or is a peptide, a proteine, a polysaccharide, a vaccine, a DNA, a RNA, an enzyme, an antibody or a fragment thereof, a hormone or an oligonucleotide, or a mixture of the above-mentioned pharmaceutically active compound,
wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,
wherein in a further embodiment the pharmaceutically active compound comprises at least one peptide for the treatment and/or prophylaxis of diabetes mellitus or complications associated with diabetes mellitus such as diabetic retinopathy, wherein in a further embodiment the pharmaceutically active compound comprises at least one human insulin or a human insulin analogue or derivative, glucagon-like peptide (GLP-1) or an analogue or derivative thereof, or exendin-3 or exendin-4 or an analogue or derivative of exendin-3 or exendin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-Y-glutamyl)-des(B30) human insulin; B29-N—(N-lithocholyl-Y-glutamyl)-des(B30) human insulin; B29-N-(ω-carboxyheptadecanoyl)-des(B30) human insulin and B29-N-(ω-carboxyheptadecanoyl) human insulin.

Exendin-4 for example means Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser- Ser-Gly-Ala-Pro-Pro-Pro-Ser-NH2.

Exendin-4 derivatives are for example selected from the following list of compounds:
H-(Lys)4-des Pro36, des Pro37 Exendin-4(1-39)-NH2,
H-(Lys)5-des Pro36, des Pro37 Exendin-4(1-39)-NH2,
des Pro36 Exendin-4(1-39),
des Pro36 [Asp28] Exendin-4(1-39),
des Pro36 [IsoAsp28] Exendin-4(1-39),
des Pro36 [Met(O)14, Asp28] Exendin-4(1-39),
des Pro36 [Met(O)14, IsoAsp28] Exendin-4(1-39),
des Pro36 [Trp(O2)25, Asp28] Exendin-4(1-39),
des Pro36 [Trp(O2)25, IsoAsp28] Exendin-4(1-39),
des Pro36 [Met(O)14 Trp(O2)25, Asp28] Exendin-4(1-39),
des Pro36 [Met(O)14 Trp(O2)25, IsoAsp28] Exendin-4(1-39); or
des Pro36 [Asp28] Exendin-4(1-39),
des Pro36 [IsoAsp28] Exendin-4(1-39),
des Pro36 [Met(O)14, Asp28] Exendin-4(1-39),
des Pro36 [Met(O)14, IsoAsp28] Exendin-4(1-39),
des Pro36 [Trp(O2)25, Asp28] Exendin-4(1-39),
des Pro36 [Trp(O2)25, IsoAsp28] Exendin-4(1-39),
des Pro36 [Met(O)14 Trp(O2)25, Asp28] Exendin-4(1-39),
des Pro36 [Met(O)14 Trp(O2)25, IsoAsp28] Exendin-4(1-39),
   wherein the group -Lys6-NH2 may be bound to the C-terminus of the Exendin-4 derivative;
   or an Exendin-4 derivative of the sequence
des Pro36 Exendin-4(1-39)-Lys6-NH2 (AVE0010),
H-(Lys)6-des Pro36 [Asp28] Exendin-4(1-39)-Lys6-NH2,
des Asp28 Pro36, Pro37, Pro38Exendin-4(1-39)-NH2,
H-(Lys)6-des Pro36, Pro38 [Asp28] Exendin-4(1-39)-NH2,
H-Asn-(Glu)5des Pro36, Pro37, Pro38 [Asp28] Exendin-4(1-39)-NH2,
des Pro36, Pro37, Pro38 [Asp28] Exendin-4(1-39)-(Lys)6-NH2,
H-(Lys)6-des Pro36, Pro37, Pro38 [Asp28] Exendin-4(1-39)-(Lys)6-NH2,
H-Asn-(Glu)5-des Pro36, Pro37, Pro38 [Asp28] Exendin-4(1-39)-(Lys)6-NH2,
H-(Lys)6-des Pro36 [Trp(O2)25, Asp28] Exendin-4(1-39)-Lys6-NH2,
H-des Asp28 Pro36, Pro37, Pro38 [Trp(O2)25] Exendin-4(1-39)-NH2,
H-(Lys)6-des Pro36, Pro37, Pro38 [Trp(O2)25, Asp28] Exendin-4(1-39)-NH2,
H-Asn-(Glu)5-des Pro36, Pro37, Pro38 [Trp(O2)25, Asp28] Exendin-4(1-39)-NH2,
des Pro36, Pro37, Pro38 [Trp(O2)25, Asp28] Exendin-4(1-39)-(Lys)6-NH2,
H-(Lys)6-des Pro36, Pro37, Pro38 [Trp(O2)25, Asp28] Exendin-4(1-39)-(Lys)6-NH2,
H-Asn-(Glu)5-des Pro36, Pro37, Pro38 [Trp(O2)25, Asp28] Exendin-4(1-39)-(Lys)6-NH2,
H-(Lys)6-des Pro36 [Met(O)14, Asp28] Exendin-4(1-39)-Lys6-NH2,
des Met(O)14 Asp28 Pro36, Pro37, Pro38 Exendin-4(1-39)-NH2,
H-(Lys)6-desPro36, Pro37, Pro38 [Met(O)14, Asp28] Exendin-4(1-39)-NH2,
H-Asn-(Glu)5-des Pro36, Pro37, Pro38 [Met(O)14, Asp28] Exendin-4(1-39)-NH2,
des Pro36, Pro37, Pro38 [Met(O)14, Asp28] Exendin-4(1-39)-(Lys)6-NH2,
H-(Lys)6-des Pro36, Pro37, Pro38 [Met(O)14, Asp28] Exendin-4(1-39)-(Lys)6-NH2,
H-Asn-(Glu)5 des Pro36, Pro37, Pro38 [Met(O)14, Asp28] Exendin-4(1-39)-(Lys)6-NH2,
H-Lys6-des Pro36 [Met(O)14, Trp(O2)25, Asp28] Exendin-4(1-39)-Lys6-NH2,
H-des Asp28 Pro36, Pro37, Pro38 [Met(O)14, Trp(O2)25] Exendin-4(1-39)-NH2,
H-(Lys)6-des Pro36, Pro37, Pro38 [Met(O)14, Asp28] Exendin-4(1-39)-NH2,
H-Asn-(Glu)5-des Pro36, Pro37, Pro38 [Met(O)14, Trp(O2)25, Asp28] Exendin-4(1-39)-NH2,
des Pro36, Pro37, Pro38 [Met(O)14, Trp(O2)25, Asp28] Exendin-4(1-39)-(Lys)6-NH2,
H-(Lys)6-des Pro36, Pro37, Pro38 [Met(O)14, Trp(O2)25, Asp28] Exendin-4(S1-39)-(Lys)6-NH2,
H-Asn-(Glu)5-des Pro36, Pro37, Pro38 [Met(O)14, Trp(O2)25, Asp28] Exendin-4(1-39)-(Lys)6-NH2;
   or a pharmaceutically acceptable salt or solvate of any one of the afore-mentioned Exendin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Antibodies are globular plasma proteins (˜150 kDa) that are also known as immunoglobulins which share a basic structure. As they have sugar chains added to amino acid residues, they are glycoproteins. The basic functional unit of each antibody is an immunoglobulin (Ig) monomer (containing only one Ig unit); secreted antibodies can also be dimeric with two Ig units as with IgA, tetrameric with four Ig units like teleost fish IgM, or pentameric with five Ig units, like mammalian IgM.

The Ig monomer is a “Y”-shaped molecule that consists of four polypeptide chains; two identical heavy chains and two identical light chains connected by disulfide bonds between cysteine residues. Each heavy chain is about 440 amino acids long; each light chain is about 220 amino acids long. Heavy and light chains each contain intrachain disulfide bonds which stabilize their folding. Each chain is composed of structural domains called Ig domains. These domains contain about 70-110 amino acids and are classified into different categories (for example, variable or V, and constant or C) according to their size and function. They have a characteristic immunoglobulin fold in which two β sheets create a “sandwich” shape, held together by interactions between conserved cysteines and other charged amino acids.

There are five types of mammalian Ig heavy chain denoted by α, δ, ε, γ, and μ. The type of heavy chain present defines the isotype of antibody; these chains are found in IgA, IgD, IgE, IgG, and IgM antibodies, respectively.

Distinct heavy chains differ in size and composition; α and γ contain approximately 450 amino acids and δ approximately 500 amino acids, while μ and ε have approximately 550 amino acids. Each heavy chain has two regions, the constant region (C_{H}) and the variable region (V_{H}). In one species, the constant region is essentially identical in all antibodies of the same isotype, but differs in antibodies of different isotypes. Heavy chains γ, α and δ have a constant region composed of three tandem Ig domains, and a hinge region for added flexibility; heavy chains μ and ε have a constant region composed of four immunoglobulin domains. The variable region of the heavy chain differs in antibodies produced by different B cells, but is the same for all antibodies produced by a single B cell or B cell clone. The variable region of each heavy chain is approximately 110 amino acids long and is composed of a single Ig domain.

In mammals, there are two types of immunoglobulin light chain denoted by λ and κ. A light chain has two successive domains: one constant domain (CL) and one variable domain (VL). The approximate length of a light chain is 211 to 217 amino acids. Each antibody contains two light chains that are always identical; only one type of light chain, K or A, is present per antibody in mammals.

Although the general structure of all antibodies is very similar, the unique property of a given antibody is determined by the variable (V) regions, as detailed above. More specifically, variable loops, three each the light (VL) and three on the heavy (VH) chain, are responsible for binding to the antigen, i.e. for its antigen specificity. These loops are referred to as the Complementarity Determining Regions (CDRs). Because CDRs from both VH and VL domains contribute to the antigen-binding site, it is the combination of the heavy and the light chains, and not either alone, that determines the final antigen specificity.

An “antibody fragment” contains at least one antigen binding fragment as defined above, and exhibits essentially the same function and specificity as the complete antibody of which the fragment is derived from. Limited proteolytic digestion with papain cleaves the Ig prototype into three fragments. Two identical amino terminal fragments, each containing one entire L chain and about half an H chain, are the antigen binding fragments (Fab). The third fragment, similar in size but containing the carboxyl terminal half of both heavy chains with their interchain disulfide bond, is the crystalizable fragment (Fc). The Fc contains carbohydrates, complement-binding, and FcR-binding sites. Limited pepsin digestion yields a single F(ab′)2 fragment containing both Fab pieces and the hinge region, including the H—H interchain disulfide bond. F(ab′)2 is divalent for antigen binding. The disulfide bond of F(ab′)2 may be cleaved in order to obtain Fab′. Moreover, the variable regions of the heavy and light chains can be fused together to form a single chain variable fragment (scFv).

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

It will be further apparent to those skilled in the art that various modifications and variations can be made to the injection device without departing from the spirit and scope of what is disclosed herein. Further, it is to be noted, that any reference numerals used in the appended claims are not to be construed as limiting the scope of the disclosure.

### BRIEF DESCRIPTION OF THE DRAWINGS

In the following, embodiments of the drive mechanism and the injection device are described in detail by making reference to the drawings, in which:

FIG. 1 is a schematic representation of the injection device comprising a dose tracker and a preselector,

FIG. 2 shows a configuration with the preselector in a first preselection positional state and the dose tracker in a first positional state and a corresponding configuration of the indicator assembly,

FIG. 3 corresponds to FIG. 2 with the dose tracker in the maximum dose positional state,

FIG. 4 is another configuration of the preselector in a second preselection positional state and the dose tracker in a first positional state smaller than the maximum dose positional state,

FIG. 5 corresponds to FIG. 4 with the dose tracker in the maximum dose positional state,

FIG. 6 shows another schematic example of an injection device,

FIG. 7 shows another example of the injection device connected or coupled with an add-on device,

FIG. 8 is a block diagram of an add-on device,

FIG. 9 is an exploded view of an injection device as handed out to a patient,

FIG. 10 is an exploded view of the components that make up the injection device according to FIG. 9,

FIG. 11 is a perspective view of another example of the injection device comprising a preselector and an indicator assembly,

FIG. 12 is a perspective enlarged view of the proximal end of the device according to FIG. 11,

FIG. 13 is a perspective illustration of the device according to FIGS. 11 and 12 with the housing partially cut away,

FIG. 14 is an isolated view of a preselector,

FIG. 15 is a longitudinal cut through the proximal end of the housing of the injection device according to FIGS. 11-13,

FIG. 16 is a side view of a proximal portion of the injection device with the preselector in a minimum preselection positional state,

FIG. 17 is the device according to FIG. 16 with the preselector in an intermediate preselection positional state, and

FIG. 18 is a side view of the injection device according to FIGS. 16 and 17 with the preselector in a maximum preselection positional state,

FIG. 19 is an isolated and perspective view of the dose tracker,

FIG. 20 is another example of a proximal end of an injection device comprising an electronically implemented indicator assembly in an initial configuration with the preselector in a minimum preselection positional state,

FIG. 21 is the device according to FIG. 20 with the dose tracker and the dose dial in the maximum dose positional state,

FIG. 22 is the device according to FIGS. 20 and 21 with the preselector in a maximum preselection positional state and with the dose dial and the dose tracker in a zero dose configuration,

FIG. 23 is another configuration of the device according to FIG. 22 with the dose tracker and the dose dialin a maximum dose positional state,

FIG. 24 is a perspective view of the injection device according to FIGS. 20-21 with the housing partially cut away,

FIG. 25 shows the indicator assembly of the device according to FIGS. 20-24 detached from the housing of the injection device,

FIG. 26 is another illustration of the injection device with the indicator assembly provided in an add-on device detached from the injection device,

FIG. 27 is a bottom view of the add-on device,

FIG. 28 is an enlarged illustration of the proximal portion of the device according to FIG. 24,

FIG. 29 shows the interaction between a first position sensor with the first marker on the dose tracker with the dose tracker in an initial configuration,

FIG. 30 represents another configuration of the position sensor according to FIG. 29 with the dose tracker in a first positional state,

FIG. 31 shows the dose tracker in a second positional state and

FIG. 32 shows the dose tracker in a third positional state.

### DETAILED DESCRIPTION

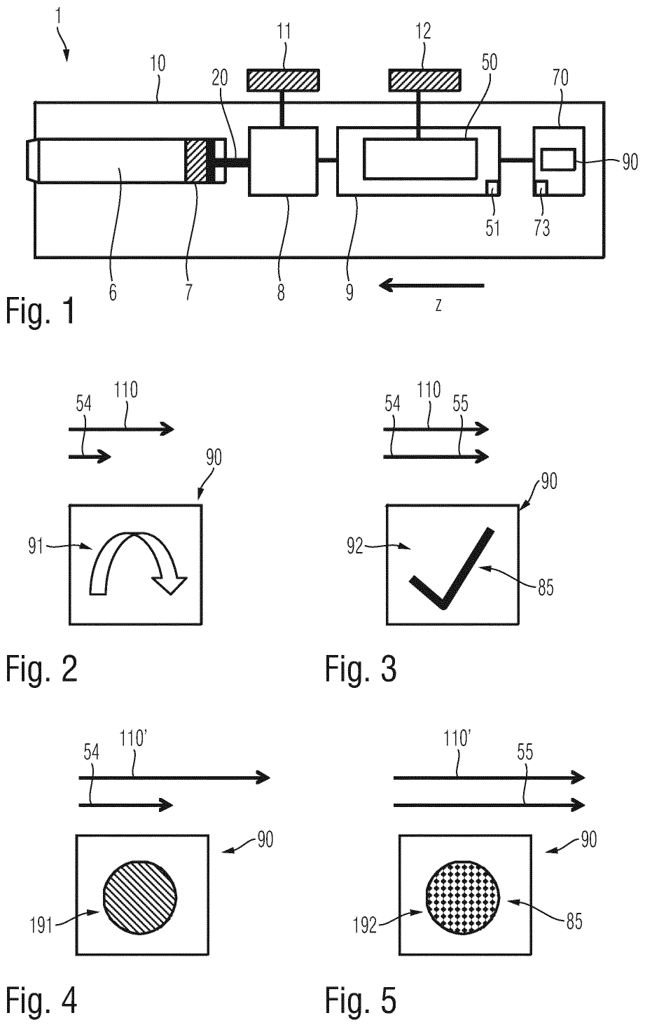

The schematic illustration of an injection device 1 in accordance to FIG. 1 is rather simplified and is only used to reveal and show the mutual interaction between a dose dial, a dose tracker, a preselector and an indicator assembly of an injection device. The injection device 1 comprises a housing 10. The housing 10 may be an elongated housing and may extend in a longitudinal or axial direction (z). Inside the housing 10 there is typically provided a reservoir or a cartridge 6 configured to contain and to hold a liquid medicament. The housing 10 comprises a distal end 2 near a dispensing end of the cartridge 6. Opposite the distal end the cartridge 6 is provided with a bung 7 that seals an interior of the cartridge 6 in proximal direction. For expelling or dispensing of a dose of the medicament from the interior of the cartridge 6 the injection device 1 comprises a drive mechanism 8. The drive mechanism 8 comprises a piston rod 20 configured to exert a distally directed pressure to the bung and in order to drive the bung 7 in distal direction.

The injection device 1 also comprises a dose setting mechanism 9. The dose setting mechanism comprises at least a dose tracker 50. The dose tracker 50 is displaceable relative to the housing 10. It is at least one of translationally or rotationally displaceable relative to the housing 10 during setting of a dose. Typically and during dose dispensing under the action of the drive mechanism 8 the dose tracker 50 returns into a zero dose configuration. In a subsequent dose setting procedure the dose tracker 50 can be become repeatedly subject to at least one of a translational or rotational displacement relative to the housing 10. The displacement of the dose tracker 50 during setting of a dose is controllable or inducible by means of a dose dial 12 that is displaceable relative to the housing 10 for setting of the dose. For instance the dose dial 12 is rotationally supported on a proximal end of the housing 10.

Insofar the illustration of FIG. 1 does not reflect the relative positions of the various components of the injection device relative to each other. The injection device 1 further comprises a trigger 11 by way of which a dose dispensing action can be triggered and/or controlled after a dose of appropriate size has been set in a dose setting procedure. The trigger is operable engageable with the drive mechanism for dispensing or expelling of the dose.

In addition to the dose tracker 50 the injection device 1 comprises a preselector 70. The preselector 70 is configured to define a maximum dose positional state 55 of the dose tracker relative to the housing 10. The maximum dose positional state 55 is that positional state of the dose tracker 50 that is furthest away from a zero dose positional state of the dose tracker. The maximum dose positional state 55 defines the maximum sized dose of a medicament that can be dispensed with the injection device.

By means of the preselector 70 the maximum dose positional state 55 and hence the maximum dose to be dispensed with the injection device 1 can be varied and modified. Insofar the preselector 70 provides a limiter or is a limiter for the dose setting mechanism 9. The dose setting mechanism 9 and the dose tracker 50 may be originally configured and designed to provide a selection of doses of variable size. With the preselector 70 only one or a few predefined doses among a range of differently sized doses that could be originally dispensed with the injection device 1 are pre-set. Hence, by means of the preselector 70 the capability of the dose setting mechanism 9 to set and to select numerous differently sized doses is reduced to only one or a few differently sized doses of the medicament.

Insofar and by means of the preselector 70 the injection device 1 can be transformed or transferred into a kind of a fixed dose injection device. This is of particular benefit for situations where the user himself should not decide about the size of a dose of the medicament to be administered. From a user perspective the preselector therefore facilitates overall usage and handling of the injection device.

Typically, the preselector 70 and the dose tracker 50 are configured to mechanically engage. The dose tracker 50 may comprise at least one tracking stop feature 51 and the preselector may comprise at least one preselector stop feature 73. By means of the preselector stop feature 73 the preselector 70 mechanically engages with the correspondingly shaped tracking stop feature 51 of the dose tracker 50. In this way a displacement of the dose tracker 50 beyond the maximum dose positional 55 state can be blocked and prevented. The preselector 70 may be only operable by caregivers or medical staff. It may be protected against misuse and may not be operable or actuatable or reconfigurable by the end user or patient. However and according to an individual prescription schedule or medication schedule the care giver may individually modify or configure the preselector 70 so that the injection device becomes capable to dispense and to expel a dose of required size.

The injection device 1 further comprises an indicator assembly 90 by way of which at least a relative position of the dose tracker 50 relative to the preselector 70 can be indicated. Since the device 1 is limited to expel or to dispense only a single sized dose of known size the injection device 1 may be void of a specific dose indicating mechanism, e.g. illustrating the dose size in terms of units of the medicament, such as international units of insulin. It is not required for the indicator assembly 90 to visualize or illustrate a number of units of the medicament actually set. Since the injection device can be limited or restricted to the dispensing or expelling of multiple doses of equal size that particular size is known to at least the caregiver. It is hence sufficient for the patient when the indicator assembly has a limited function and is only capable to provide a comparison between the actual positional state 54 of the dose tracker compared to the predefined maximum dose positional state 55 of the dose tracker 50.

In FIG. 2 a configuration is illustrated wherein the preselector 70 is in a first preselection positional state 110. The length of the arrow representing the first preselection positional state 110 is indicative of the path length the dose dial 12 or the dose tracker 50 may be displaced starting from a zero dose configuration until reaching a maximum dose positional state 55. In the configuration according to FIG. 2 the momentary positional state 54 of the dose tracker 50 is also illustrated. This arrow is shorter than the arrow representing the first preselection positional state 110. Accordingly, the indicator assembly 90 as shown in FIG. 2 provides an indication 91 that the maximum dose positional state 55 has not yet been reached. For example the indication 91 is illustrated as a curved arrow indicating to a user of the injection device to dial the dose dial 12 further in a dose incrementing direction, e.g. clockwise.

In the configuration according to FIG. 3 the dose dial 12 and hence the dose tracker 50 have been further displaced. Here, the positional state 54 of the dose tracker 50 coincides and corresponds to the maximum dose positional state 55. Accordingly the indicator assembly 90 provides a first marker 85, e.g. in form of a second indication 92 that represents a confirmation that the dose tracker 50 has reached the maximum dose positional state 55.

The illustration of FIG. 4 is somewhat comparable to the configuration of FIG. 2. The configuration as illustrated in FIG. 5 is somewhat comparable to the configuration of FIG. 3. In FIG. 4 the preselector 70 has been moved to a second preselection positional state 110′. In comparison to FIG. 2 the second preselection positional state 110′ represents a larger maximum dose of the medicament to be expelled. Also here, the reduced length of the arrow representing the actual positional state 54 of the dose tracker 50 illustrates that the dose tracker 50 has not yet reached the maximum dose positional state 55. Accordingly, the indicator assembly 90 provides a first indicator 191 that differs from a second indicator 192 as illustrated in FIG. 5. Also here, the second indicator 192 represents a first marker 85 that is indicative that the dose tracker 50 has reached the maximum dose positional state 55. The indicators 90, 91, 191, 192 may comprise symbols, numbers, letters or just a differently colored structure in order to distinguish between the maximum dose positional state of the dose tracker and a non-maximum dose positional state of the dose tracker.

With the present injection device 1 injection and hence dispensing of the dose should only be triggered and commence when the dose tracker has reached the maximum dose positional state 55.

The preselector 70 is at least one of translationally or rotationally displaceable relative to the housing 10 between the at least two preselection positional states 110, 110′. In any of the available preselection positional states 110, 110′ the preselector is lockable relative to the housing 10. Hence during dose setting the preselector 70 is stationary or fixed to the housing 10 while the dose tracker 50 is subject to at least one of a translational or rotational displacement relative to the housing 10.

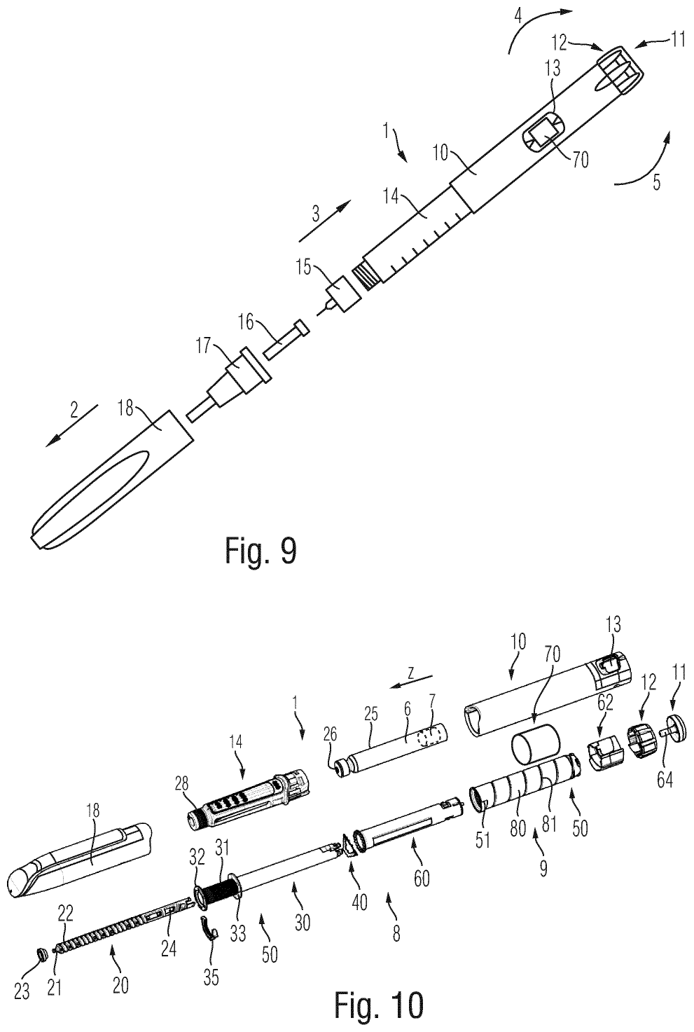

The schematic illustration of the injection device 1 directly corresponds to the implementation of an injection device as described later with regard to FIGS. 9-19. Here, the indicator assembly 90 is integrated or connected to the preselector 70. The dose tracker is provided with at least a first marker 85. The indicator assembly 90 is configured to reveal the first marker 85 on the dose tracker 50 when the dose tracker 50 reaches the maximum dose positional state 55. In the maximum dose positional state 55 the relative position or orientation of the first marker coincides or aligns with the position of the indicator assembly 90. In this way and when reaching the maximum dose positional state 55 the at least first marker 85 is revealed by the indicator assembly 90. For instance, the indicator assembly 90 may comprise an aperture 75 through which aperture the dose tracker or at least a portion thereof, namely a portion provided with the first marker 85 is discernible.

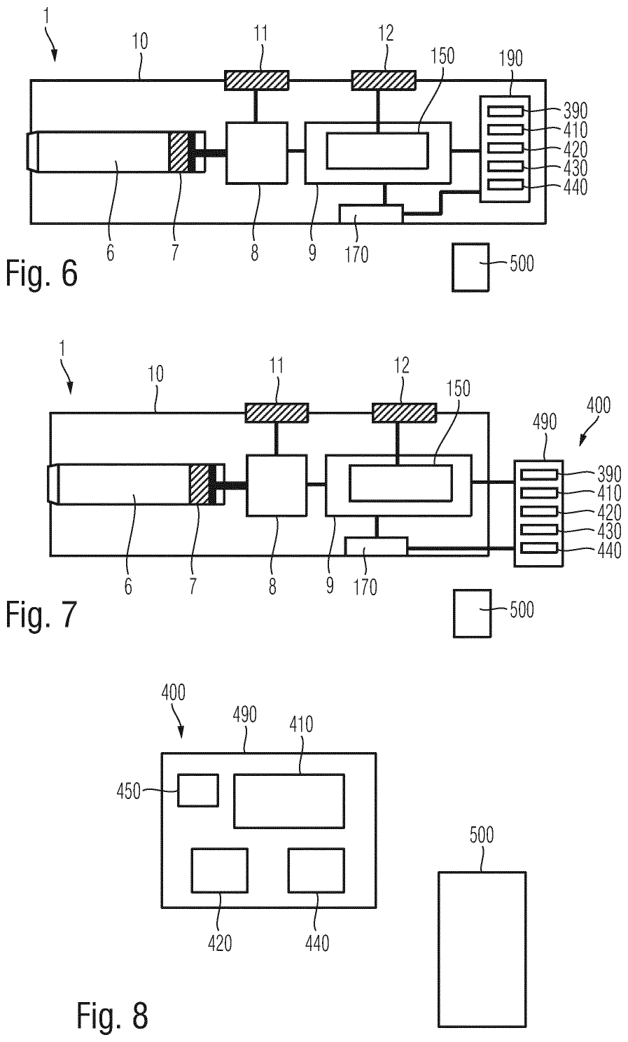

In the schematic representation of FIG. 6 the indicator assembly 190 is implemented as an electronic indicator assembly. The indicator assembly 190 may interact separately with the dose tracker 150 and with the preselector 170. For instance, the indicator assembly 190 may be configured to determine the preselection positional state 110 of the preselector 170 separate from the positional state 54 of the dose tracker 150. Here, the indicator assembly 190 does not have to be integrated or connected to the preselector 170. The indicator assembly 190 may be located elsewhere in or on the housing 10 of the injection device 1.

Typically, the indicator assembly 190 comprises a first position sensor 430 configured to determine or to detect a positional state of the dose tracker 150. The indicator assembly 190 may comprise a second position sensor 390 configured to determine or to detect a positional state of the preselector 170. Moreover, the electronically implemented indicator assembly 190 may comprise a processor 420 to process signals obtainable from the first position sensor 390 and obtainable from the second position sensor 430.

The processor 420 may be configured to compare the positional state of the dose tracker 150. If the processor 420 determines that the positional state of the dose tracker 150 corresponds to the maximum dose positional state 55 as governed and defined by the momentary preselection positional state 110 of the preselector 70 the processor 420 is configured go provide a respective indicator on an electronic display 410 of the indicator assembly 190. The indicator assembly 190 may further comprise a communication unit 440, e.g. implemented in form of an antenna to receive and/or to transmit electronic data or signals. By means of the communication unit 440 the indicator assembly 190 may exchange data with an external electronic device 500.

The external electronic device 500 may be implemented as a smartphone. The external electronic device may comprise a portable electronic device. It may be provided with a software or a software application that is configured to communicate with the communication unit 440 in a wireless way.

The drive mechanism 8 and the dose setting mechanism 9 as well as the preselector 170 and the dose tracker 150 do not have to be implemented all mechanically. At least one of the drive mechanism 8, the dose setting mechanism 9, the dose tracker 150 and the preselector 170 may be implemented electromechanically. In this way the indicator assembly 190 or its processor 420 can be configured to reconfigure the preselector 170. For instance, the external electronic device 500 may be configured to communicate with the processor 420 via the communication unit 440 to modify the configuration of the preselector 170. When implemented as an electromechanical dose setting mechanism 9 the preselector 170 may be electronically implemented and may be reconfigurable remotely by means of at least one of the electronic device 500 and the processor 420.

In FIG. 7 another embodiment of the injection device 1 is illustrated. Here, the indicator assembly 190 is not integrated into the device 1 but is provided as a remote indicator assembly 490 that is provided in or on an add-on device 400. The add-on device 400 is configured for detachable attachment to the housing 10 of the injection device 1. The add-on device 400 comprises all those features and components of the electronic indicator assembly 190 as described above in connection with FIG. 6. The detachable add-on device 400 is particularly configured and dedicated for use with injection devices 1 that are configured as disposable injection devices and which are intended to be discarded entirely once the medicament has been used up.

In FIG. 8 a rather schematic illustration of the add-on device 400 is provided. The add-on device 400 comprises the remote indicator assembly 490 that is configured to visualize or to indicate audibly or haptically to a user that a maximum dose positional state 55 of the dose tracker 150 has been reached. The electronic display 410 may comprise a so called electronic paper or e-paper display device that mimics the appearance of ordinary ink on paper. The electronic display 410 may comprise one of an electrophoretic display, a micro encapsulated electrophoretic display, an electrowetting display, an electrofluidic display and a plasmonic electronic display. Such electronic paper displays only consume a minimum of electric energy.

The communication unit 440 may comprise an antenna, such as an RFID antenna. The external electronic device 500 may comprise an RFID reader configured to wirelessly provide and to wirelessly transfer electric energy to the remote indicator assembly 490. Alternative or additional the communication unit 440 may comprise other radio frequency based antennas that may be configured to support one of a WLAN, Wi-Fi or Bluetooth communication protocol. In this way the processor 420 and the electronic display 410 may receive sufficient power for visualizing or for generally indicating the positional state of the dose tracker 150 relative to the preselector 170. The add-on device 400 and/or the remote indicator assembly 490 may also comprise at least one actuation element 450, in form of at least one or several buttons. The actuation element 450 may be also integrated into the electronic display 410. Here, the electronic display 410 may comprise a touch sensitive display.

By means of the actuation element 450 a user may confirm that a dose dispensing operation has just been conducted. Also during dispensing or during dose setting the user may press on the actuation element 450 thus inducing a change of the visual appearance of the electronic display 410. In this way, the electronic display 410 may alternately provide information regarding the preselection positional state and a momentary positional state of the dose tracker.

In the following an example of a handheld and all mechanically implemented injection device 1 is described with regard to FIGS. 9 and 10.

The injection device 1 as shown in FIGS. 9 and 10 is a pre-filled disposable injection device that comprises a housing 10 to which an injection needle 15 can be affixed. The injection needle 15 is protected by an inner needle cap 16 and either an outer needle cap 17 or a protective cap 18 that is configured to enclose and to protect a distal section of the housing 10 of the injection device 1. The housing 10 may comprise and form a main housing part configured to accommodate a drive mechanism 8 as shown in FIG. 10. The injection device 1 may further comprise a distal housing component denoted as cartridge holder 14. The cartridge holder 14 may be permanently or releasably connected to the main housing 10. The cartridge holder 14 is typically configured to accommodate a cartridge 6 that is filled with a liquid medicament. The cartridge 6 comprises a cylindrically-shaped or tubular-shaped barrel 25 sealed in proximal direction 3 by means of a bung 7 located inside the barrel 25. The bung 7 is displaceable relative to the barrel 25 of the cartridge 6 in a distal direction 2 by means of a piston rod 20. A distal end of the cartridge 6 is sealed by a pierceable seal 26 configured as a septum and being pierceable by a proximally directed tipped end of the injection needle 15. The cartridge holder 14 comprises a threaded socket 28 at its distal end to threadedly engage with a correspondingly threaded portion of the injection needle 15. By attaching the injection needle 15 to the distal end of the cartridge holder 14 the seal 26 of the cartridge 6 is penetrated thereby establishing a fluid transferring access to the interior of the cartridge 6.

When the injection device 1 is configured to administer e.g. human insulin, the dosage set by a dial 12 at a proximal end of the injection device 1 may be displayed in so-called international units (IU, wherein 1 IU is the biological equivalent of about 45.5 μg of pure crystalline insulin (1/22 mg).

As shown further in FIGS. 9 and 10, the housing 10 comprises a dosage window 13 that may be in the form of an aperture in the housing 10. The dosage window 13 permits a user to view a limited portion of a number sleeve 80 that is configured to move when the dial 12 is turned, to provide a visual indication of a currently set dose. The dial 12 is rotated on a helical path with respect to the housing 10 when turned during setting and/or dispensing or expelling of a dose.

The injection device 1 may be configured so that turning the dosage knob 12 causes a mechanical click sound to provide acoustical feedback to a user. The number sleeve 80 mechanically interacts with a piston in the insulin cartridge 6. When the needle 15 is stuck into a skin portion of a patient, and when the trigger 11 or injection button is pushed, the insulin dose displayed in display window 13 will be ejected from injection device 1. When the needle 15 of the injection device 1 remains for a certain time in the skin portion after the trigger 11 is pushed, a high percentage of the dose is actually injected into the patient's body. Ejection of an insulin dose may also cause a mechanical click sound, which is however different from the sounds produced when using the dial 12.

In this embodiment, during delivery of the insulin dose, the dial 12 is turned to its initial position in an axial movement, that is to say without rotation, while the number sleeve 80 is rotated to return to its initial position, e.g. to display a dose of zero units.

The injection device 1 may be used for several injection processes until either the cartridge 6 is empty or the expiration date of the medicament in the injection device 1 (e.g. 28 days after the first use) is reached.

Furthermore, before using injection device 1 for the first time, it may be necessary to perform a so-called “prime shot” to remove air from the cartridge 6 and the needle 15, for instance by selecting two units of the medicament and pressing trigger 11 while holding the injection device 1 with the needle 15 upwards. For simplicity of presentation, in the following, it will be assumed that the ejected amounts substantially correspond to the injected doses, so that, for instance the amount of medicament ejected from the injection device 1 is equal to the dose received by the user.

The expelling or drive mechanism 8 as illustrated in more detail in FIG. 10 comprises numerous mechanically interacting components. A flange like support of the housing 10 comprises a threaded axial through opening threadedly engaged with a first thread or distal thread 22 of the piston rod 20. The distal end of the piston rod 20 comprises a bearing 21 on which a pressure foot 23 is free to rotate with the longitudinal axis of the piston rod 20 as an axis of rotation. The pressure foot 23 is configured to axially abut against a proximally facing thrust receiving face of the bung 7 of the cartridge 6. During a dispensing action the piston rod 20 rotates relative to the housing 10 thereby experiencing a distally directed advancing motion relative to the housing 10 30 and hence relative to the barrel 25 of the cartridge 6. As a consequence, the bung 7 of the cartridge 6 is displaced in distal direction 2 by a well-defined distance due to the threaded engagement of the piston rod 20 with the housing 10.

The piston rod 20 is further provided with a second thread 24 at its proximal end. The distal thread 22 and the proximal thread 24 are oppositely handed.

There is further provided a drive sleeve 30 having a hollow interior to receive the piston rod 20. The drive sleeve 30 comprises an inner thread threadedly engaged with the proximal thread 24 of the piston rod 20. Moreover, the drive sleeve 30 comprises an outer threaded section 31 at its distal end. The threaded section 31 is axially confined between a distal flange portion 32 and another flange portion 33 located at a predefined axial distance from the distal flange portion 32. Between the two flange portions 32, 33 there is provided a last dose limiter 35 in form of a semi-circular nut having an internal thread mating the threaded section 31 of the drive sleeve 30.

The last dose limiter 35 further comprises a radial recess or protrusion at its outer circumference to engage with a complementary-shaped recess or protrusion at an inside of the sidewall of the housing 10. In this way the last dose limiter 35 is splined to the housing 10. A rotation of the drive sleeve 30 in a dose incrementing direction 4 or clockwise direction during consecutive dose setting procedures leads to an accumulative axial displacement of the last dose limiter 35 relative to the drive sleeve 30. There is further provided an annular spring 40 that is in axial abutment with a proximally facing surface of the flange portion 33. Moreover, there is provided a tubular-shaped clutch 60. At a first end the clutch 60 is provided with a series of circumferentially directed saw teeth. Towards a second opposite end of the clutch 60 there is located a radially inwardly directed flange.

Furthermore, there is provided a dose dial sleeve also denoted as number sleeve 80. The number sleeve 80 is provided outside of the spring 40 and the clutch 60 and is located radially inward of the housing 10. A helical groove 81 is provided about an outer surface of the number sleeve 80. The housing 10 is provided with the dosage window 13 through which a part of the outer surface of the number 80 can be seen. The housing 10 is further provided with a helical rib at an inside sidewall portion of an insert piece 62, which helical rib is to be seated in the helical groove 81 of the number sleeve 80. The tubular shaped insert piece 62 is inserted into the proximal end of the housing 10. It is rotationally and axially fixed to the housing 10. There are provided first and second stops on the housing 10 to limit a dose setting procedure during which the number sleeve 80 is rotated in a helical motion relative to the housing 10. As will be explained below in greater detail, at least one of the stops is provided by a preselector stop feature 73 provided on a preselector 70.

The dose dial 12 in form of a dose dial grip is disposed about an outer surface of the proximal end of the number sleeve 80. An outer diameter of the dose dial 12 typically corresponds to and matches with the outer diameter of the housing 10. The dose dial 12 is secured to the number 80 to prevent relative movement therebetween. The dose dial 12 is provided with a central opening.

The trigger 11, also denoted as dose button is substantially T-shaped. It is provided at a proximal end of the injection device 10. A stem 64 of the trigger 11 extends through the opening in the dose dial 12, through an inner diameter of extensions of the drive sleeve 30 and into a receiving recess at the proximal end of the piston rod 20. The stem 64 is retained for limited axial movement in the drive sleeve 30 and against rotation with respect thereto. A head of the trigger 11 is generally circular. The trigger side wall or skirt extends from a periphery of the head and is further adapted to be seated in a proximally accessible annular recess of the dose dial 12.

To dial a dose a user rotates the dose dial 12. With the spring 40 also acting as a clicker and the clutch 60 engaged, the drive sleeve 30 the spring or clicker 40, the clutch 60 and the number sleeve 80 rotate with the dose dial 12. Audible and tactile feedback of the dose being dialed is provided by the spring 40 and by the clutch 60. Torque is transmitted through saw teeth between the spring 40 and the clutch 60. The helical groove 81 on the number sleeve 80 and a helical groove in the drive sleeve 30 have the same lead. This allows the number sleeve 80 to extend from the housing 10 and the drive sleeve 30 to climb the piston rod 20 at the same rate. At a limit of travel a radial stop on the number sleeve 80 engages either with a first stop or a second stop provided on the housing 10 provided on the pre-selector 70 to prevent further movement in a dose incrementing direction 4. Rotation of the piston rod 20 is prevented due to the opposing directions of the overall and driven threads on the piston rod 20.

The last dose limiter 35 keyed to the housing 10 is advanced along the threaded section 31 by the rotation of the drive sleeve 30. When a final dose dispensed position is reached, a radial stop formed on a surface of the last dose limiter 35 abuts a radial stop on the flange portion 33 of the drive sleeve 30, preventing both, the last dose limiter 35 and the drive sleeve 30 from rotating further.

Should a user inadvertently dial beyond the desired dosage, the injection device 1, configured as a pen-injector allows the dosage to be dialed down without dispense of the medicament from the cartridge 6. For this the dose dial 12 is simply counter-rotated. This causes the system to act in reverse. A flexible arm of the spring or clicker 40 then acts as a ratchet preventing the spring 40 from rotating. The torque transmitted through the clutch 60 causes the saw teeth to ride over one another to create the clicks corresponding to dialed dose reduction. Typically, the saw teeth are so disposed that a circumferential extent of each saw tooth corresponds to a unit dose.

When the desired dose has been dialed the user may simply dispense the set dose by depressing the trigger 11. This displaces the clutch 60 axially with respect to the number sleeve 80 causing dog teeth thereof to disengage. However, the clutch 60 remains keyed in rotation to the drive sleeve 30. The number sleeve 80 and the dose dial 12 are now free to rotate in accordance with the helical groove 81.

The axial movement deforms the flexible arm of the spring 40 to ensure the saw teeth cannot be overhauled during dispense. This prevents the drive sleeve 30 from rotating with respect to the housing 10 though it is still free to move axially with respect thereto. The deformation is subsequently used to urge the spring 40 and the clutch 60 back along the drive sleeve 30 to restore the connection between the clutch 60 and the number sleeve 80 when the distally directed dispensing pressure is removed from the trigger 11.

The longitudinal axial movement of the drive sleeve 30 causes the piston rod 20 to rotate through the through opening of the support of the housing 10, thereby to advance the bung 7 in the cartridge 6. Once the dialed dose has been dispensed, the number sleeve 80 is prevented from further rotation by contact of at least one stop extending from the dose dial 12 with at least one corresponding stop of the housing 10. A zero dose position may be determined by the abutment of one of axially extending edges or stops of the number sleeve 80 with at least one or several corresponding stops of the housing 10.

The expelling mechanism or drive mechanism 8 as described above is only exemplary for one of a plurality of differently configured drive mechanisms that are generally implementable in a disposable pen-injector. The drive mechanism as described above is explained in more detail e.g. in WO2004/078239A1, WO 2004/078240A1 or WO 2004/078241A1 the entirety of which being incorporated herein by reference.

The injection device 1 further comprises a preselector 70 that is configured to define a maximum dose positional state 55 of the number sleeve 80. Here, the number sleeve 80 represents or substitutes the dose tracker 50 as described above. Although and in view of the indicator assembly 90 the number sleeve is no longer required to illustrate dose indicating numbers in the dosage window 13 of the housing 10. The dose tracker 50 and hence the number sleeve 80 comprises a tracking stop feature 51 to engage with a correspondingly shaped preselector stop feature 73 as will be described in greater detail with regard to FIG. 13.

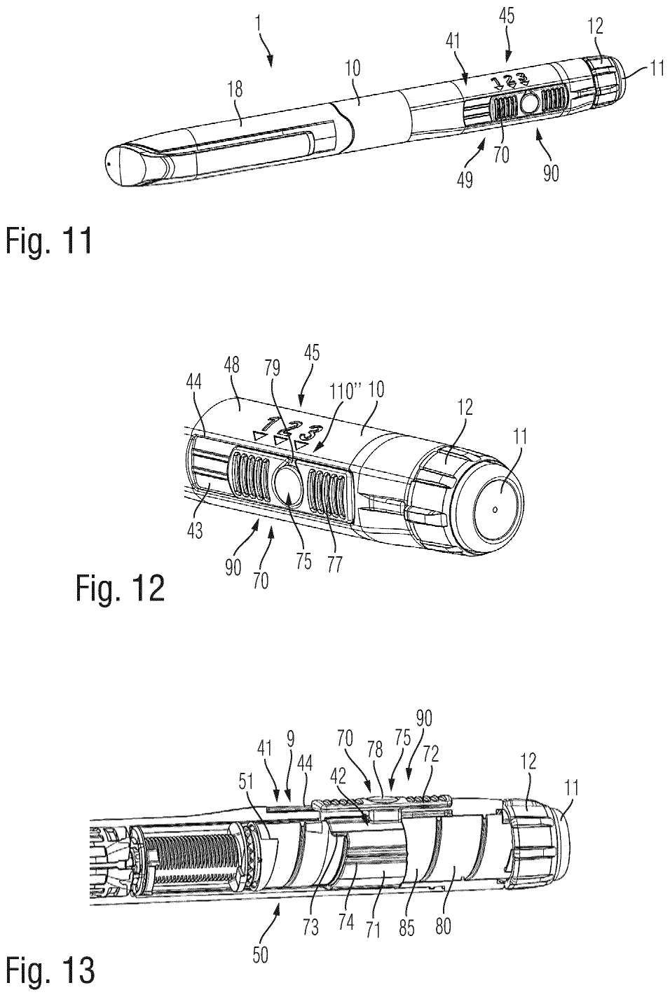

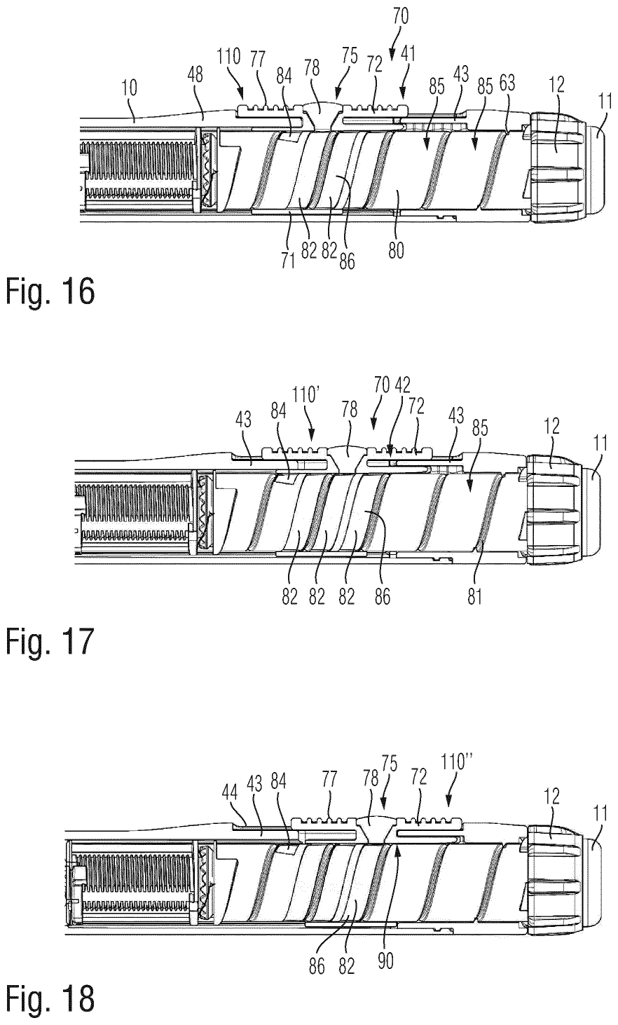

As shown in FIGS. 11-13 the dose setting mechanism 9 comprises a preselector 70 that is at least one of translationally or rotationally displaceable relative to the housing 10 and hence relative to the sidewall 48 of the housing 10 between at least two preselection positional states 110, 110′ and 110″ as illustrated in the sequence of FIGS. 16, 17 and 18. The preselector 70 behaves as a limiter and provides a limiter function in order to delimit a displacement of the dose tracker 50 relative to the housing 10. The preselector 70 comprises a sleeve portion 71 enclosing at least a longitudinal section of the dose tracker 50 and hence of the number sleeve 80. Near an axial end the sleeve portion 71 comprises a preselector stop feature 73 in the form of an axial protrusion with a side edge extending parallel to the longitudinal axis of the housing 10.

The dose tracker 50 comprises a correspondingly shaped tracking stop feature 51. As shown in FIG. 13 there is a predefined axial distance between the tracking stop feature 51 and the preselector stop feature 73. The preselector 70 is hindered from rotation relative to the housing 10. It comprises a spline feature 74, which is in the form of a longitudinal protrusion received in a correspondingly shaped recess on an inside facing section of the sidewall 48 of the housing 10. In this way the preselector 70 is permanently rotationally locked to the housing 10. With the spline feature 74 the preselector 70 and its sleeve portion 71 is free to slide in axial or longitudinal direction relative to the housing 10. For providing a keyed engagement between the sleeve portion 71 and the housing 10 also the housing 10 may comprise a radially inwardly extending protrusion to slide in a groove or slot on the outside surface of the sleeve portion 71.

The preselector 70 further comprises a slider 72 that is accessible from outside the housing 10. The slider 72 can be integrally formed with the sleeve portion 71. The sleeve portion 71 and the slider 72 may be also provided as separate pieces that are connected together by mutually corresponding connectors, not explicitly illustrated here. The slider 72 is axially guided along a displacement path 49 formed by a recess 41 on an outside facing surface portion of the sidewall 48 of the housing 10. The recess 41 may be circumferentially confined by a surrounding edge 44 forming a stepped down portion in the outside surface of the sidewall 48. The recess 41 comprises a through opening 42 as indicated in FIG. 13. The through opening 42 is formed in a bottom section 43 of the recess. The through opening 42 comprises a longitudinal slit through which the interconnection of the sleeve portion 71 and the slider 72 extends. On the outside surface the slider 72 has a gripping structure 77 featuring various protrusions that are separated in axial direction so as to provide a rather slip-free engagement, e.g. with a finger of a hand of a user.

The preselector 70 further comprises a detent structure 76 best shown in FIG. 15. The detent structure 76 is configured to engage with a correspondingly shaped counter detent structure 46 provided on the sidewall 48 of the housing 10. The counter detent structure 46 comprises at least two recesses 47 that are separated from each other along the longitudinal axis. The detent structure 76 of the preselector 70 comprises a resiliently deformable snap feature, such as a radially deformable tongue to mechanically engage with one of the recesses 47 of the counter detent structure 46 of the housing 10. In this way the preselector 70 is displaceable relative to the housing between at least two preselection positional states 110, 110′, 110″. In any of the preselection positional states 110, 110′, 110″ the preselector 70 is locked to the housing. In order to displace the preselector 70 relative to the housing, e.g. from one preselection positional state 110 to another preselection positional state 110′ the holding force acting between and provided by the detent structure 76 and the counter detent structure 46 must be overcome.

The axial position of the preselector 70 determines a maximum distance the dose tracker 50 can be displaced starting from a zero dose configuration as shown in FIG. 13, 16, 17 or 18 to a maximum dose configuration in which the dose tracker 50 protrudes from a proximal end of the housing 10 until the tracking stop feature 51 abuts with the preselector stop feature 73 thus blocking any further proximally directed and dose increasing rotation of the dose tracker 50 in unison with the dose dial 12.

The dose tracker 50 and hence the number sleeve 80 comprises a helical groove 81 engaged with a correspondingly shaped protrusion 63 provided at the proximal end of the housing 10. The protrusion 63 may be provided on an inside facing surface portion of the insert 62 fixed to the proximal end of the housing 10. The insert facilitates assembly of the components inside the housing of the injection device.

Also a portion of the bottom section 43 and hence of the recess 41 may be provided by the insert 62. The insert 62 may provide a proximal end cap of the housing 10 of the injection device 1.

The preselector 70 is arrestable or fixable to the sidewall 48 of the housing 10 in at least two different discrete positions denoted as preselection positional states 110, 110′, 110″. The axial distance between neighboring preselection positional states 110, 110′ is identical and corresponds to the longitudinal advancing motion of the dose tracker 50 as the dose tracker 50 undergoes a complete revolution relative to the housing 10. In this way it is guaranteed, that the tracking stop feature 51 always exactly engages with the preselector stop feature 73 when reaching the maximum dose positional state 55.

In the configuration as shown in FIG. 16 the dose tracker 50 can be rotated at least by one complete revolution until its tracking stop feature 51 abuts and engages with the preselector stop feature 73. By displacing the preselector 70 to the next preselection positional state 110 as shown in FIG. 17, the dose tracker 50 can be rotated at least by two complete revolutions until the tracking stop feature 51 and the preselector stop feature 73 mechanically engage thereby blocking any further dose increasing displacement of the dose tracker 50 and hence of the dose dial 12.

In the configuration of FIG. 18 the preselector 70 is in the most proximal position of three discrete allowable and supported preselection positional states 110, 110′, 110″. In this position of the preselector 70 the dose tracker 50 can be rotated at least by three complete revolutions relative to the housing 10.

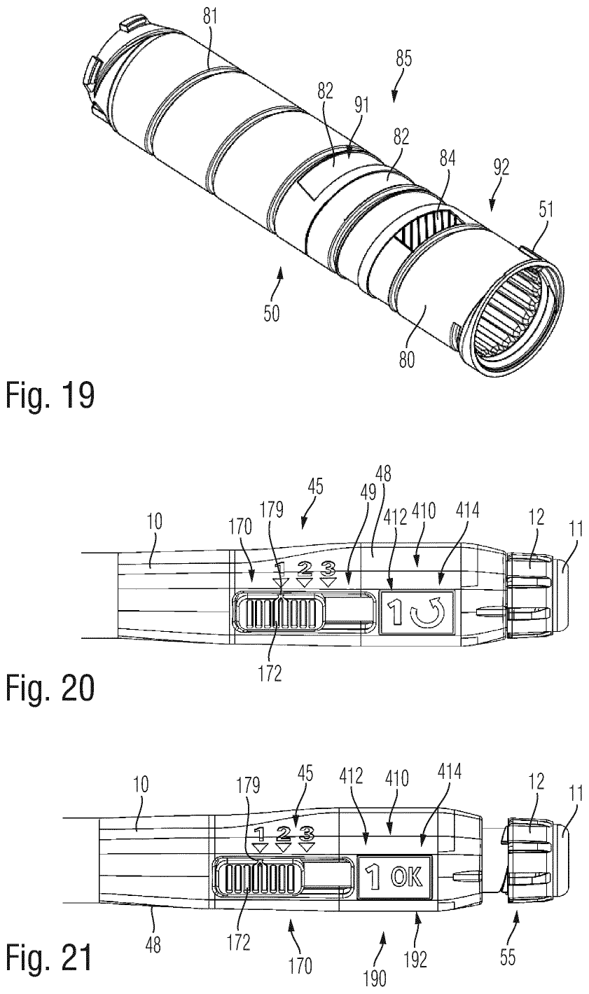

In FIG. 19 an isolated view of the dose tracker 50 in form of the number sleeve 80 is provided. As shown there the outside surface of the dose tracker 50 comprises a first marker 85. The first marker 85 comprises a first surface section 82 that may be provided with a first indication 91. The marker 85 comprises a second surface section 84 non-overlapping with the first surface section 82. The second surface section 84 may be provided with a second indication 92. As already described in connection with FIGS. 2 and 3 first and second indications 91, 92 may comprise different symbols or different colors.

The second surface section 84 and hence the second indication 92 may provide a confirmation symbol indicating to the end user that the dose tracker 50 has reached the predefined maximum dose positional state 55. The second surface section 84 and the second indication 92 are arranged in such a distance to the tracking stop feature 51 which distance substantially corresponds to the longitudinal distance between the preselector stop feature 73 and an aperture 75 provided in the preselector 70. The aperture 75 extends radially through the preselector 70. The aperture 75 may extend through an overlapping portion of the sleeve portion 71 and the slider 72. As illustrated in FIG. 16 the aperture 75 may be provided with a magnifying lens 78.

In this way and when reaching the maximum dose positional state 55 in which the tracking stop feature 51 engages with the preselector stop feature 73 the second surface portion 84 and hence the second indication 92 overlaps with and aligns with the aperture 75 so that the second surface portion 84 and the second indication 92 provided thereon becomes discernible and visible through the aperture 75. This is an indication to the end user that the maximum dose positional state 55 has been reached.

Before reaching the maximum dose positional state 55 the first surface portion 82, eventually with the first indication 91 is revealed in the aperture 75 thus indicating to the user that the maximum dose positional state 55 has not yet been reached and that dispensing should not yet commence.

On an outside surface of the sidewall 48 of the housing 10 there is further provided at least one preselection indication 45. Apparently and as illustrated in FIGS. 11 and 12 there are provided three consecutive preselection indications 45. The preselection indications 45 comprise a triangular shaped pointer in combination with a number, e.g. 1, 2, 3. The preselection indications 45 are arranged along the displacement path 49 of the preselector 70 and hence of its slider 72. Near or aligned with the aperture 75 there is further provided a pointer 79. Since the preselection indications 45 are separated along the displacement path 49 of the preselector 70 in any one of the preselection positional states 110, 110′, 110″ the pointer 79 points to one of the preselection indications 45. The preselection indication 45 in the present embodiment corresponds to the total number of revolutions of the dose tracker 50 for the respective preselection positional state. However, the preselection indication 45 may also represent standard units of the medicament to be dispensed or may provide any other dose size indicating symbol.

In the sketches of FIGS. 11 and 12 the preselector 70 is in the largest preselection positional state 110″. Accordingly, the pointer 79 is in alignment with the largest preselection indication 45, namely with number 3.

The position of the preselector 70 relative to the housing 10 is therefore indicative of the maximum dose that can be set with the dose setting mechanism 9. The indicator assembly 90 presently integrated into the preselector 70 is then indicative, that the momentary configuration and status of the dose setting mechanism 9 corresponds to the pre-set dose.

Even though the presently illustrated embodiments only show a fixing or locking of the limiter 70 or the preselector 70 at discrete positions relative to the housing 10 that correspond to consecutive and complete revolutions of the dose tracker 50 it is also conceivable that the dose tracker 50 comprises two or even three tracking stop features 51 to engage with the limiter stop feature or preselector stop feature 73. Alternatively also the preselector 70 may comprise two or more preselector stop feature 73 to engage with the tracking stop feature 51. In this way the maximum dose positional state could be assigned with every half or every third revolution of the dose tracker 50 relative to the housing 10. Furthermore it is conceivable, that two or more tracking stop feature 51 simultaneously engage with correspondingly shaped two or more preselector stop feature 73. In this way the mechanical interaction and robustness of the abutment between the dose tracker 50 and the preselector 70 can be enhanced and increased.

FIGS. 20-24 show another example of an injection device. Here and contrary to the injection device 1 as described in connection with FIGS. 11-19 the indicator assembly 190 is implemented as an electronic indicator assembly. As shown in FIG. 20 the electronic indicator assembly 190 comprises an electronic display 410 having a first display section 412 and having a second display section 414. The display 410 is typically provided on or near a proximal end of the housing 10. The preselector 170 is all mechanically implemented as already described in connection with FIGS. 11-19. Also here the preselector 170 comprises a sleeve portion 171 that is rotationally locked to the housing 10 by means of a spline feature 174 on an outside surface of the sleeve portion 171.

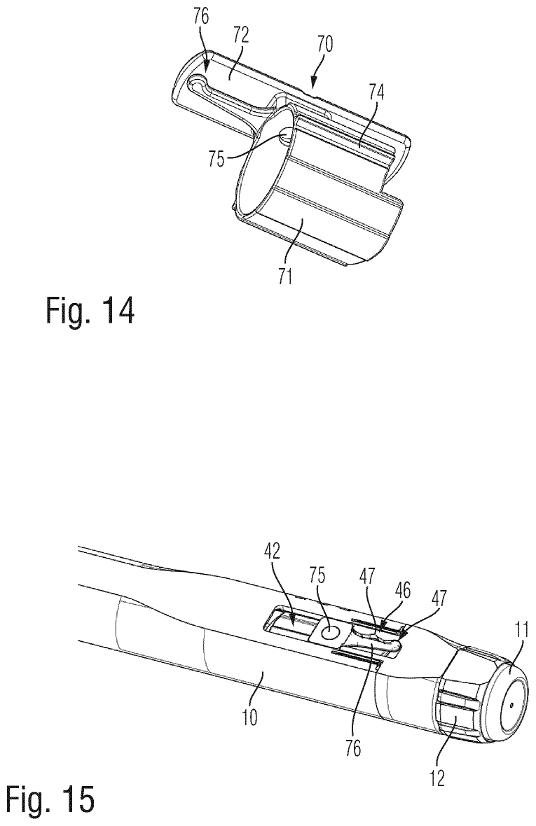

The sleeve portion 171 encloses a portion of an outside surface of the dose tracker 150. Also here the dose tracker 150 comprises a number sleeve 180. The dose tracker 150 also comprises a tracking stop feature 151 to abut and to engage with a correspondingly shaped preselector limiting stop feature 173. The sleeve portion 171 is connected to a slider 172 having a gripping structure 177 on an outside facing surface section. The slider also comprises a pointer 179 to point to one of the three preselection indications 45 that are arranged along the displacement path 49 of the slider 172. Even though not illustrated in detail the preselector 70 and the housing 10 comprise a detent structure 76 and a counter detent structure 46 as described in connection with FIGS. 14 and 15 above.

In this way the preselector 170 can be fixed and locked to the sidewall 48 of the housing 10 in any of the available preselection positional states 110, 110′, 110″.

Contrary to the example as shown in FIGS. 11-19 the indicator assembly 190 is provided separate from the preselector 170. For this the indicator assembly 190 comprises a first position sensor 430 and a second position sensor 390 as illustrated in FIG. 27. The first position sensor 430 is configured to determine or to detect a positional state of the dose tracker 150 relative to the housing 10. The second position sensor 390 is configured to determine and to detect a preselection positional state of the preselector 70. Both position sensors 430, 390 are configured to generate electric signals being indicative of the detected position of the dose tracker 150 and the preselector 170.

The indicator assembly 190 further comprises a processor 420 connected to the first position sensor 430 and connected to the second position sensor 390. In this way the indicator assembly 190 is enabled to compare a measured or detected positional state 54 of the dose tracker 150 with the preselection positional state 110 of the preselector 170. Depending on the comparison the electronically implemented indicator assembly 190 is configured to provide one of at least two indicators 191, 192 as shown in FIGS. 20 and 21. In FIG. 20 in which the dose tracker is not yet in the maximum dose positional state 55 the indicator assembly 190 provides a first indication 191 in the second display section 414 thus illustrating to the user that the dose dial 12 has to be rotated further.

If the dose dial 12 has been rotated further so that the dose tracker 50 is screwed out of the housing 10 in proximal direction the tracking stop feature 151 engages with the preselector stop feature 173. The position of the dose tracker 150 detected by the indicator assembly 190 then reveals that the maximum dose positional state 55 has been reached. Accordingly, the indicator assembly 190 is configured to provide the second indication 192 in the second display section 414 of the display 410 thereby confirming to the user that the dose tracker 150 has reached the intended maximum dose positional state 55. In the configuration as shown in FIG. 21 the injection device 1 is ready for starting a dispensing operation.

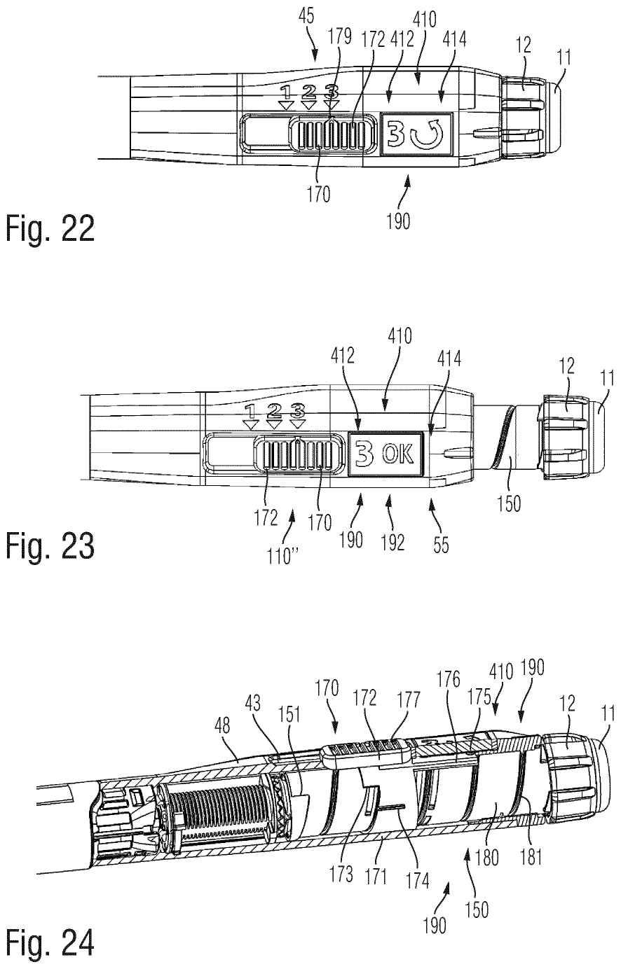

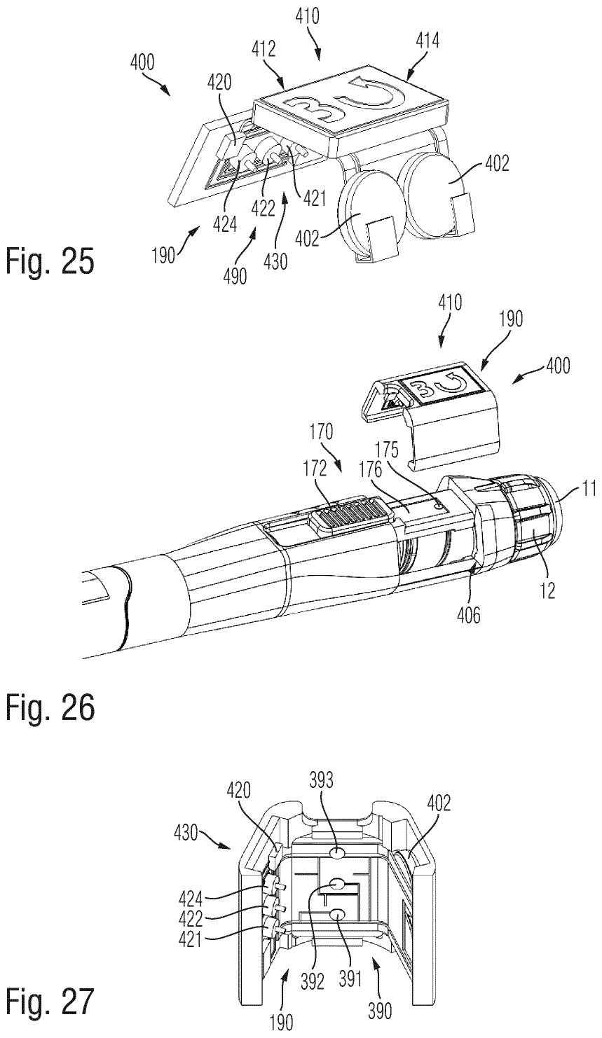

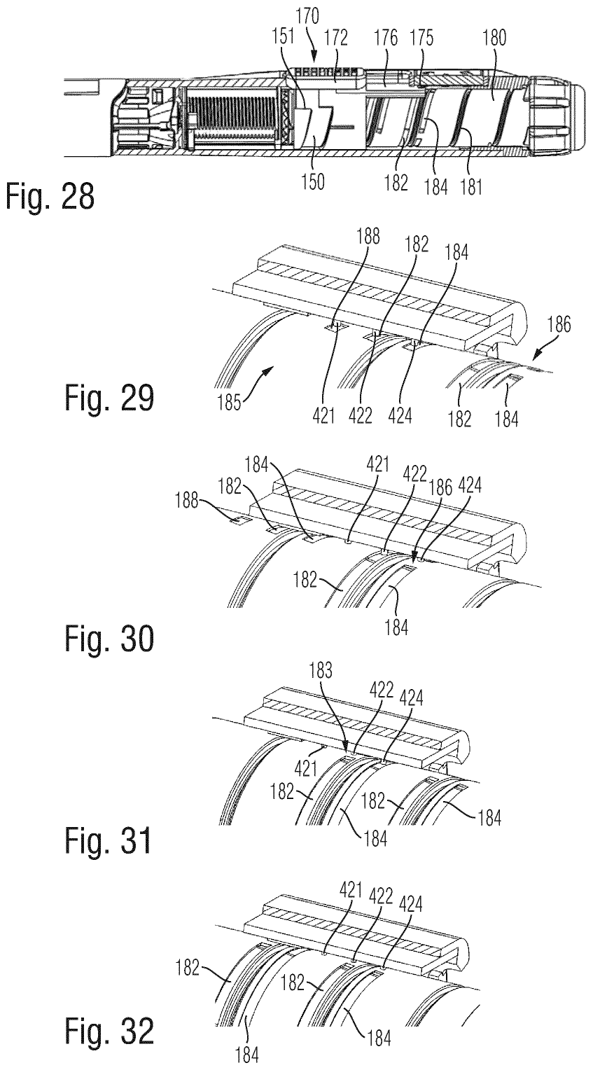

In the first display section 412 there is provided the preselection indication 45 to which the pointer 179 of the slider 172 actually points. In the configuration of FIGS. 20 and 21 the pointer 179 is in alignment with the smallest preselection indication 445 of three available preselection indications 45. In the configuration of FIGS. 22 and 23 the preselector 170 has been displaced to a different preselection positional state 110″ compared to the preselection positional state 110 as shown in FIG. 20. Correspondingly, in the first display section 412 the preselection indication 45 coinciding with the pointer 179 is reproduced. In a zero dose configuration in which the dose dial 12 and hence the dose tracker 150 have not yet been dialed or displaced in a dose incrementing direction the second display section 414 provides the first indication 191 as described above thereby indicating that the maximum dose positional state has not yet been reached.

By rotating and displacing the dose tracker 50 relative to the housing 10 along the dose incrementing direction 4 until the maximum dose positional state 55 has been reached, as shown in FIG. 23, the electronic display 410 of the indicator assembly 190 switches to the second indication 192 thereby confirming that the maximum dose positional state 55 has been reached. The device is then ready to commence and to conduct a dose dispensing procedure.

The preselector 170 comprises an extension 176. In the present illustration the extension 176 is radially overlapping at least with a portion of the electronic indicator assembly 190. The extension 176 is provided with a second marker 175. The second marker 175 is configured to interact with the sensor sections 391, 392, 393 of the second position sensor 390 as shown in FIG. 27. The second marker 175 is provided on an outside facing surface of the preselector 170. In any of the available preselection positional states 110, 110′, 110″ the second marker 175 is in contact or is aligned with only one of the sensor sections 391, 392, 393. In the preselection positional state 110 representing the smallest maximum dose size the second marker 175 may be aligned with the sensor section 393.

In an intermediate preselection positional state 110′ the second marker 175 is aligned with the sensor section 392. In the preselection positional state 110″ representing a maximum dose the second marker 175 is aligned with the sensor section 391. The second marker 175 may be electrically conducting. Its presence or vicinity to any one of the sensor sections 391, 392, 393 can be electronically detected by means of the processor 420 of the indicator assembly 190 being electrically connected to the position sensor 390.

In a similar way the first position sensor 430 may comprise a first detector element 422 and a second detector element 424. The detector elements 422 424 are arranged axially offset. They may be implemented as electrical contacts or as electromechanical switches, which upon mechanical contact with another structure or component may generate or interrupt an electrical signal that is transferrable to the processor 420. On an outside surface of the number sleeve 180 and hence on an outside surface of the dose tracker 150 there are provided two surface sections, namely a first surface section 182 and a second surface section 184. The surface sections 182, 184 comprise the shape of a helical thread.

The lead of the helical threads of the first and second surface sections 182, 184 is identical to a lead of a helical groove 181 of the dose tracker 150 or the number sleeve 180. As the dose tracker 150 is subject to a helical motion relative to the sidewall 48 of the housing 10 the first detector element 422 slides along the first surface section 182. Likewise, the second detector element 424 slides along the second surface section 184.

In the zero dose configuration as shown in FIG. 29 both detector elements 422, 424 are in contact with the respective first and second surface sections 182, 184. The outside surface of the number sleeve 180 and hence of the dose tracker 150 comprises at least a first marker 185 by way of which the first position sensor 430 becomes enabled to determine and to detect at least one of an axial or rotational position of the dose tracker 150. The marker 185 comprises at least two interrupts 183, 186 in the first surface section 182 and in the second surface section 184, respectively. In the illustration of FIGS. 29-33 the distal direction points to the right whereas the proximal direction points to the left.

As illustrated in FIG. 31 the first surface section 182 comprises a first interrupt 183. As illustrated in FIG. 30 the second surface section 184 comprises a second interrupt 186. After a complete revolution of the dose tracker 150 and hence of the number sleeve 180 the second interrupt 186 is aligned with the second detector element 424 as illustrated in FIG. 30. In this way, the second detector element 424 is switched. The second detector element 424 when aligned with the interrupt 186 may generate or interrupt an electrical signal that differs from an electrical signal of the second detector element 424 when aligned with the second surface section 184.

After two revolutions the opposite situation occurs as illustrated in FIG. 31. There, the second detector element 424 is in alignment and is in contact with the second surface section 184 while the first detector element 422 is in alignment with the first interrupt 183 of the first surface section 182. This configuration may represent a logical 2 while the configuration of FIG. 30 may represent a logical 1. In FIG. 32 and after three complete revolutions of the dose tracker 150, none of the detector elements 422, 424 remains in contact with any of the first or second surface sections 182, 184. This configuration may represent a logical 3. Hence, with two detector elements 424, 424 and with two surface sections 182, 184 each of which comprising at least one interruption 183, 186 four different discrete states of the dose tracker 150 can be electronically detected and determined.

The detector elements 421, 422, 424 can be implemented as mechanical switches when the surface sections 382, 384 and the interrupts 183, 186 comprise different radial height or depth on the outside surface of the 150. For instance, the surface sections 382, 384 each comprise a longitudinal groove on the outside surface of the number sleeve 180. The interrupts 183, 186 may flush with the outside surface of the number sleeve 180 or of the dose tracker 150. When implemented as mechanical switches, the detector elements 422, 424 may each comprise a radially displaceable and spring biased pin running along the respective surface section 182, 184 as the number sleeve 180 is subject to a rotation relative to the housing 10. When a pin of one of the detector elements 422, 424 aligns with an interrupt 183, 186 the respective pin is depressed against the action of the spring. Such a depression is accompanied by a closing or opening of an electrical switch or contact inside the detector element 422, 424.

There is further illustrated an optional detector element 421 that serves as an on-off switch for the add-on device 400. The further detector element 421 is configured to engage with a further surface section 188 on the outside surface of the number sleeve 80. The surface section 188 as illustrated in FIGS. 29 and 30 comprises a confined recess in the outside surface of the number sleeve 180 or of the dose tracker 150. The recess and hence the surface section 188 is only slightly larger than the extent of the detector element 421. In an initial configuration or zero-dose configuration as for instance illustrated in FIG. 29 the detector element 421 is in alignment with the recess at the surface section 188.

As soon as the number sleeve 180 is subject to a rotation relative to the housing 10 the detector element 421 and the recess 188 get out of engagement. Consequently, the detector element 421 will be subject to a radially outwardly directed depression as it starts to slide outside the recessed surface section 188 and hence along the outside surface of the number sleeve 180. In this way the add-on device 400 and hence the electronic components thereof, in particular its processor 420, is switched on and the status of the further detector elements 422, 424 can be monitored and processed. By means of a detector element 421 implemented as on/off switch, electric energy consumption of the add-on device 400 can be reduced and battery lifetime can be prolonged.

When after completion of a dose dispensing procedure the detector element 421 re-engages the recessed surface section 188 the add-on device 400 is switched off and electric energy can be saved.

The first position sensor 430 and the second position sensor 390 can be implemented in many different ways. Here, the first and second surface sections 182, 184 may be provided as radial grooves while the interrupts 183, 186 protrude radially from the respective groove. Alternatively, the interrupts 183, 186 can be provided as non-conductive sections on respective first and second electrically conductive surface sections 182, 184; or vice versa. Alternatively, the first and/or second surface sections 182, 184 could be electrically or magnetically encoded. For instance, the surface sections 182, 184 may be electrically conductive while the interrupts 183, 186 are electrically insulating or non-conductive. It is also conceivable that the surface sections 182, 184 and the interrupts 183, 186 mutually distinguish in terms of their visual appearance or light absorption characteristic as well and/or with regards to their magnetic properties.

In this way also other encoding schemes based on an optic encoding or magnetic encoding can be generally implemented. With an optically or magnetically implemented encoding of the outside surface 85 of the dose tracker 150 or the number sleeve 180 also respective first and second detector elements 422, 424 should be implemented. Here, first and second detector elements 422, 424 may be implemented as light detectors or as magnetic sensors.

In a situation wherein the slider 172 is located near the proximal end of the displacement path and wherein a maximum dose has been pre-selected, i.e. with the pointer 179 of the slider 172 aligns with the preselection indication 45 provided with number 3 the configurations as shown in FIGS. 29, 30 and 31 correspond to positions of the dose tracker 150 and hence of the number sleeve 180 that are smaller than the pre-selected dose size. Only when arriving in the proximal end position, in which the preselector stop feature 173 engages with the tracking stop feature 151 the electronic position detection of the dose tracker 150 coincides with the electronic position detection of the preselector 170. In such a situation the processor 420 is configured to switch the indicator 415 in the second display section 414 to confirm that the intended dose size has been set.

These electronic components may belong to the injection device 1 and hence to the dose setting mechanism 9 thereof. The injection device 1 may be further equipped with one or several electric power sources 402, such as button batteries. The electric power sources 402 can be integrated into the housing 10 or can be detachably mounted inside the housing 10 and/or its sidewall 48.

In a further embodiment and as indicated in FIG. 26 the electronic components as well as the electric power sources 402 can be all integrated into an add-on device 400 that may be detachably connectable to the housing 10, in particular to its sidewall 48. A detachable add-on device 400 may be usable with disposable injection devices that are equipped with a pre-filled cartridge 6 and which are intended to be discarded entirely after the content of the cartridge 6 has been used up.

Here, the injection device 1 could be void of an own indicator assembly 190. The add-on device 400 can be provided with a remote indicator assembly 490 which has the same structure as the indicator assembly 190 as described above. Here, all components belonging to the indicator assembly 190 are provided on the detachable add-on device 400, which may have a fastener or fastening structure 404 to engage with a correspondingly shaped counter fastener 406 in order to guarantee a correct and well-defined position of the add-on device 400 relative to the housing 10, to the dose tracker 150 and to the preselector 170.

The add-on device 400 is further equipped with the above mentioned first and second position sensors 430, 390 configured to detect and to determine a position of the dose tracker 150 relative to the preselector 170.

List of reference numbers

| | |
|---|---|
| 1 | injection device |
| 2 | distal direction |
| 3 | proximal direction |
| 4 | dose incrementing direction |
| 5 | dose decrementing direction |
| 6 | cartridge |
| 7 | bung |
| 8 | drive mechanism |
| 9 | dose setting mechanism |
| 10 | housing |
| 11 | trigger |
| 12 | dial |
| 13 | dosage window |
| 14 | cartridge holder |
| 15 | injection needle |
| 16 | inner needle cap |
| 17 | outer needle cap |
| 18 | protective cap |
| 20 | piston rod |
| 21 | bearing |
| 22 | first thread |
| 23 | pressure foot |
| 24 | second thread |
| 25 | barrel |
| 26 | seal |
| 28 | threaded socket |
| 30 | drive sleeve |
| 31 | threaded section |
| 32 | flange |
| 33 | flange |
| 35 | last dose limiter |
| 36 | shoulder |
| 40 | spring |
| 41 | recess |
| 42 | through opening |
| 43 | bottom section |
| 44 | edge |
| 45 | preselection indication |
| 46 | counter detent structure |
| 47 | recess |
| 48 | sidewall |
| 49 | displacement path |
| 50 | dose tracker |
| 51 | tracking stop feature |
| 54 | positional state |
| 55 | maximum dose positional state |
| 60 | clutch |
| 62 | insert |
| 63 | protrusion |
| 64 | stem |
| 70 | preselector |
| 71 | sleeve portion |
| 72 | slider |
| 73 | preselector stop feature |
| 74 | spline feature |
| 75 | aperture |
| 76 | detent structure |
| 77 | gripping structure |
| 78 | lens |
| 79 | pointer |
| 80 | number sleeve |
| 81 | groove |
| 82 | surface section |
| 84 | surface section |
| 85 | marker |
| 90 | indicator assembly |
| 91 | indication |
| 92 | indication |
| 110 | preselection positional state |
| 150 | dose tracker |
| 151 | tracking stop feature |
| 170 | preselector |
| 171 | sleeve portion |
| 172 | slider |
| 173 | preselector stop feature |
| 174 | spline feature |
| 175 | marker |
| 176 | extension |
| 177 | gripping structure |
| 179 | pointer |
| 180 | number sleeve |
| 181 | groove |
| 182 | surface section |
| 183 | interrupt |
| 184 | surface section |
| 185 | marker |
| 186 | interrupt |
| 188 | surface section |
| 190 | indicator assembly |
| 191 | indication |
| 192 | indication |
| 390 | position sensor |
| 391 | sensor section |
| 392 | sensor section |
| 393 | sensor section |
| 400 | add-on device |
| 402 | electric power source |
| 404 | fastener |
| 406 | counter fastener |
| 410 | electronic display |
| 412 | display section |
| 414 | display section |
| 420 | processor |
| 421 | detector element |
| 422 | detector element |
| 424 | detector element |
| 430 | position sensor |
| 440 | communication unit |
| 450 | actuation element |
| 490 | remote indicator assembly |
| 500 | external electronic device |

1. An injection device for setting and injecting a dose of a medicament, the injection device comprising:
a housing;
a dose setting mechanism arranged in the housing;
a dose dial displaceable relative to the housing for setting of the dose;
a dose tracker operably connectable to the dose dial, wherein the dose tracker is at least one of translationally displaceable or rotationally displaceable relative to the housing during setting of a dose, and wherein a positional state of the dose tracker relative to the housing is indicative of a size of the dose;
a preselector configured to define a maximum dose positional state of the dose tracker relative to the housing; and
a first marker provided on one of the dose tracker and the preselector and configured to indicate the positional state of the dose tracker relative to the preselector.

2. The injection device according to claim 1, wherein the preselector is configured to vary the maximum dose positional state of the dose tracker.

3. The injection device according to claim 1, wherein the preselector is at least one of translationally displaceable or rotationally displaceable relative to the housing between at least two preselection positional states, and wherein the preselector is lockable relative to the housing in any of the at least two preselection positional states.

4. The injection device according to claim 1, further comprising an indicator assembly configured to indicate when the dose tracker reaches the maximum dose positional state.

5. The injection device according to claim 4, wherein the first marker is located on the dose tracker, and wherein the indicator assembly is configured to reveal the first marker on the dose tracker when the dose tracker reaches the maximum dose positional state.

6. The injection device according to claim 4, wherein the indicator assembly is integrated into the preselector and comprises an aperture extending through the preselector, and wherein the first marker of the dose tracker is discernible through the aperture when the dose tracker reaches the maximum dose positional state.

7. The injection device according to claim 6, wherein the indicator assembly comprises a magnifying lens arranged in the aperture.

8. The injection device according to claim 1, wherein the dose tracker comprises at least one tracking stop feature, wherein the preselector comprises at least one preselector stop feature, and wherein the preselector stop feature is configured to engage with the at least one tracking stop feature to block and to prevent a displacement of the dose tracker beyond the maximum dose positional state.

9. The injection device according to claim 4, wherein the first marker is provided on the dose tracker, wherein a second marker is provided on the preselector, and wherein the indicator assembly is configured to determine the positional state of the dose tracker based on the first marker and to determine a preselection positional state of the preselector based on the second marker.

10. The injection device according to claim 9, wherein the indicator assembly is configured to compare the positional state of the dose tracker with the preselection positional state of the preselector.

11. The injection device according to claim 3, wherein the indicator assembly comprises a processor and an electronic display to:
visualize at least one of the positional state of the dose tracker and a preselection positional state of the preselector; and/or
visualize the positional state of the dose tracker relative to the preselection positional state of the preselector.

12. The injection device according to claim 11, wherein the indicator assembly comprises a communication unit connected to the processor to exchange electronic data with a remote electronic device.

13. The injection device according to claim 1, further comprising a piston rod and a cartridge comprising a barrel filled with a medicament.

14. An add-on device configured for attachment or coupling to an injection device for setting and injecting a dose of a medicament, the injection device comprising:
a housing,
a dose setting mechanism arranged in the housing,
a dose dial displaceable relative to the housing for setting of the dose,
a dose tracker operably connectable to the dose dial, wherein the dose tracker is at least one of translationally displaceable or rotationally displaceable relative to the housing during setting of a dose, and wherein a positional state of the dose tracker relative to the housing is indicative of a size of the dose,
a preselector configured to define a maximum dose positional state of the dose tracker relative to the housing, and
a first marker provided on one of the dose tracker and the preselector and configured to indicate the positional state of the dose tracker relative to the preselector;
wherein the add-on device comprises: a remote indicator assembly configured to indicate a positional state of the dose tracker relative to the preselector.

15. The add-on device according to claim 14, wherein the remote indicator assembly comprises at least one of:
a first position sensor configured to determine the positional state of the dose tracker, and
a second position sensor configured to determine a preselection positional state of the preselector.

16. The add-on device according to claim 14, wherein the indicator assembly comprises a processor and an electronic display configured for:
visualizing at least one of the positional state of the dose tracker and a preselection positional state of the preselector; and/or
visualizing the positional state of the dose tracker relative to the preselection positional state of the preselector.

17. The injection device according to claim 13, wherein the barrel is sealed by a bung that is axially displaceable relative to the barrel by the piston rod.

18. The add-on device according to claim 14, wherein the injection device further a cartridge comprising a barrel filled with a medicament.

19. The add-on device according to claim 18, wherein the injection device further comprises a piston rod and wherein the barrel of the cartridge is sealed by a bung that is axially displaceable relative to the barrel by the piston rod.

20. The add-on device according to claim 14, wherein the preselector of the injection device is configured to vary the maximum dose positional state of the dose tracker.
